SEQUENCE LISTING (2 sequences)

SEQ ID NO: 1 - protein, 6 aa, Artificial Sequence
  Synthetic
SFFLRN

SEQ ID NO: 2 - protein, 70 aa, Homo sapiens
EAEEDGDLQCLCVKTTSQVRPRHITSLEVIKAGPHCPTAQLIATLKNGRKICLDLQAPLYKKIIKKLLES

(19) United States
(12) Patent Application Publication
(10) Pub. No.: US 2024/0164367 A1
(43) Pub. Date: May 23, 2024

(54) METHODS FOR MAINTAINING LONG-TERM PLATELET VIABILITY AND ACTIVATABILITY

(71) Applicant: RETHAM TECHNOLOGIES, LLC, Wauwatosa, WI (US)

(72) Inventors: Anand Padmanabhan, Rochester, MN (US); Curtis Gerald Jones, Wauwatosa, WI (US)

(21) Appl. No.: 18/551,476

(22) PCT Filed: Mar. 14, 2022
(86) PCT No.: PCT/US2022/020115

§ 371 Date: Sep. 20, 2023

Related U.S. Application Data
(60) Provisional application No. 63/177,203, filed on Apr. 20, 2021.

(51) Int. Cl. A01N 1/02; A61K 35/19; C12N 5/078

(52) U.S. Cl. CPC A01N 1/0221; A61K 35/19; C12N 5/0644

(57) ABSTRACT

The compositions and methods described herein provide stabilized platelets by a controlled freezing process coupled to methods for recovery upon thawing. The stabilized platelets maintain the ability to be activated after freezing and thawing.

### CROSS-REFERENCE TO RELATED APPLICATION(S)

This application claims priority to U.S. Provisional Application No. 63/177,203, filed on Apr. 20, 2021, which is incorporated by reference herein in its entirety.

### FEDERALLY SPONSORED RESEARCH

This invention was made with government support under grant number HL147734 awarded by the National Institutes of Health Small Business Innovation Research (SBIR) program. The government has certain rights in the invention.

### REFERENCE TO SEQUENCE LISTING

This application is filed with a Computer Readable Form of a Sequence Listing in accord with 37 C.F.R. § 1.821(c). The text file submitted by EFS, “211509-9003-WO01_sequence_listing_11-MAR-2022_ST25.txt,” was created on Mar. 11, 2022, contains 2 sequences, has a file size of 1.13 Kbytes, and is hereby incorporated by reference in its entirety.

### TECHNICAL FIELD

The compositions and methods described herein provide stabilized platelets by a controlled freezing process coupled to methods for recovery upon thawing. The stabilized platelets maintain the ability to be activated after freezing and thawing.

### BACKGROUND

Platelets are anucleate fragments of megakaryocytes important for normal hemostasis. In some pathological states, thrombosis caused/contributed by activated platelets can cause high levels of morbidity and mortality. Conversely, low levels of platelets can predispose to serious bleeding. A key feature of platelets, whether used in the diagnostic or therapeutic setting, is their limited viability. Viable platelets should be able to release granule components that can be conveniently measured by techniques including but not limited to flow cytometry and enzyme-linked immunosorbent assays. Typically, platelets are stored at room temperature and become outdated in 5 days. Storing platelets at 4° C. can prolong their viability for up to 2 weeks. Other methods for long-term platelet storage, such as lyophilization, have demonstrated potential utility in treating bleeding patients, but their utility in the diagnostic setting is limited due to the fact that they are typically not viable enough to be activated and release granule components.

Methods are needed for stabilizing platelets during frozen storage that maintain their viability and activation potential in the clinical and diagnostic settings. Such stabilized platelets are useful for a variety of research purposes.

### SUMMARY

One embodiment described herein is a method for cryopreserving platelets that maintains viability and activation potential, the method comprising: (a) obtaining blood or platelet-rich plasma, or apheresis-derived platelets from a subject; (b) isolating platelets from the blood, platelet-rich plasma, or apheresis platelets; (c) suspending the platelets in a suspension buffer comprising trehalose; (d) adding a stabilizing agent to the suspended platelets; (e) cooling the platelets to a temperature of about −80° C.; and (f) storing the platelets at about −80° C. In one aspect, the method maintains the viability and activation potential of the platelets for greater than or equal to 2 weeks. In another aspect, the suspension buffer comprises about 50 mM trehalose. In another aspect, the stabilizing agent is bovine serum albumin (BSA) at about 4% by mass. In another aspect, the platelets of step (c) are diluted with suspension buffer to a final concentration of about 1×10⁶ platelets/μL. In another aspect, the diluted platelets are incubated at about 37° C. for about 2 hours with periodic agitation. In another aspect, isolating the platelets comprises centrifugation of the blood, platelet-rich plasma, or apheresis platelets at 100×g for 15 minutes to generate a first pellet and a first supernatant; followed by centrifugation of the first supernatant at 1000×g for 15 minutes to generate a second pellet and a second supernatant, wherein the first supernatant is platelet-rich and the second supernatant is platelet-poor. In another aspect, the second supernatant is collected, trehalose is added to about 50 mM, and is used to dilute the platelets of step (c) to a concentration of about 1×10⁶ platelets/μL. In another aspect, cooling the platelets comprises decreasing the temperature at a rate of about 2-20° C. per minute. Another embodiment described herein is cryopreserved platelets prepared by the methods described herein.

Another embodiment described herein is a method for cryopreserving and recovering platelets from cryopreservation, the method comprising: (a) obtaining blood, platelet-rich plasma, or apheresis-derived platelets from a subject; (b) isolating platelets from the blood, platelet-rich plasma, or apheresis platelets; (c) suspending the platelets in a buffer solution comprising trehalose; (d) adding a stabilizing agent to the suspended platelets; (e) cooling the platelets to a temperature of about −80° C.; (f) storing the platelets at about −80° C. for a period of time; and (g) thawing the platelets at about 20-40° C. In one aspect, the suspension buffer comprises about 50 mM trehalose. In another aspect, the stabilizing agent is bovine serum albumin (BSA) at about 4% by mass. In another aspect, the platelets of step (c) are diluted with suspension buffer to a final concentration of about 1×10⁶ platelets/μL. In another aspect, the diluted platelets are incubated at about 37° C. for about 2 hours with periodic agitation. In another aspect, the method further comprises the steps: (h) isolating the thawed platelets; and (i) suspending the isolated thawed platelets in an activation buffer comprising BSA at about 1% by mass. In another aspect, isolating the thawed platelets comprises centrifugation at 1000×g for 15 minutes. In another aspect, the activation buffer further comprises about 50 mM trehalose. In another aspect, isolating the thawed platelets comprises centrifugation at 1000×g for 5 minutes. In another aspect, after step (h) and prior to step (i), the following step is performed: (h-1) washing the thawed platelets in a wash buffer. In another aspect, isolating the thawed platelets in step (h) comprises centrifugation at 1000×g for 15 minutes and removal of the supernatant; and washing the thawed platelets in step (h-1) comprises resuspending platelets in wash buffer, centrifugation at 1000×g for 15 minutes and removal of supernatant. In another aspect, the wash buffer further comprises about 50 mM trehalose. In another aspect, isolating the thawed platelets in step (h) comprises centrifugation at 1000×g for 5 minutes and removal of the supernatant; and washing the thawed platelets in step (h-1) comprises resuspending platelets in wash buffer, centrifugation at 1000×g for 5 minutes and removal of supernatant. In another aspect, the period of time in step (f) is from 1 day to about 5 years. Another embodiment described herein is recovered cryopreserved platelets prepared by the methods described herein.

Another embodiment described herein is a method for activation of recovered platelets after cryopreservation, the method comprising: (a) recovering platelets after cryopreservation according to the methods described herein; (b) treating the recovered platelets with platelet factor 4 (PF4; SEQ ID NO: 2); and (c) incubating the PF4-treated platelets with a plasma, serum, or blood sample from a subject diagnosed with heparin induced thrombocytopenia (HIT). In one aspect, treating the recovered platelets with PF4 comprises adding PF4 to the platelets at a concentration of about 150 μg/mL and incubating for about 20 minutes at room temperature. In another aspect, activation of the platelets is assessed by measuring platelet granule content released after incubation of the PF4-treated platelets with the plasma, serum, or blood sample from the HIT subject. In another aspect, the platelet granule content is thrombospondin-1. Another embodiment described herein is activated recovered cryopreserved platelets prepared by the methods described herein.

Another embodiment described herein is a method for activation of recovered platelets after cryopreservation, the method comprising: (a) recovering platelets after cryopreservation according to the methods described herein; and (b) treating the recovered platelets with Thrombin Receptor Activating Peptide (TRAP; amino acid sequence: SFFLRN; SEQ ID NO: 1). In one aspect, treating the recovered platelets with TRAP comprises adding TRAP to the platelets at a concentration of about 25 μg/mL and incubating for about 30 minutes at room temperature. In another aspect, activation of the platelets is assessed by measuring platelet granule content released after treating the recovered platelets with TRAP. In another aspect, the platelet granule content is thrombospondin-1. Another embodiment described herein is activated recovered cryopreserved platelets prepared by the methods described herein.

Another embodiment described herein is a method for activation of recovered platelets after cryopreservation, the method comprising: (a) recovering platelets after cryopreservation according to the methods described herein; (b) treating the recovered platelets with heparin; and (c) incubating the heparin-treated platelets with a plasma, serum, or blood sample from a subject diagnosed with heparin induced thrombocytopenia (HIT). In one aspect, treating the recovered platelets with heparin comprises adding heparin to the platelets at a concentration of about 0.1-5 U/mL and incubating for about 20 minutes at room temperature. In another aspect, activation of the platelets is assessed by measuring platelet granule content released after incubation of the heparin-treated platelets with the plasma, serum, or blood sample from the HIT subject. In another aspect, the platelet granule content is thrombospondin-1. Another embodiment described herein is activated recovered cryopreserved platelets prepared by the methods described herein.

Another embodiment described herein is a method for cryopreserving platelets and recovering platelets from cryopreservation, the method comprising: (a) obtaining blood, platelet-rich plasma, or apheresis-derived platelets from a subject; (b) isolating platelets from the blood, platelet-rich plasma, or apheresis platelet; (c) suspending the platelets in a buffer solution comprising trehalose; (d) adding a stabilizing agent to the suspended platelets; (e) cooling the platelets to a temperature of about −80° C.; (f) storing the platelets at about −80° C. for a period of time; (g) thawing the platelets at about 20-40° C.; (h) isolating the thawed platelets; and (i) suspending the isolated thawed platelets in an activation buffer comprising BSA at about 1% by mass. In one aspect, the suspension buffer comprises about 50 mM trehalose. In another aspect, the stabilizing agent is bovine serum albumin (BSA) at about 4% by mass. In another aspect, the platelets of step (c) are diluted with suspension buffer to a final concentration of about 1×10⁶ platelets/μL. In another aspect, the diluted platelets are incubated at about 37° C. for about 2 hours with periodic agitation. In another aspect, isolating the thawed platelets comprises centrifugation at 1000×g for 15 minutes. In another aspect, the activation buffer further comprises about 50 mM trehalose. In another aspect, isolating the thawed platelets comprises centrifugation at 1000×g for 5 minutes. In another aspect, after step (h) and prior to step (i), the following step is performed: (h-1) washing the thawed platelets in a wash buffer. In another aspect, isolating the thawed platelets in step (h) comprises centrifugation at 1000×g for 15 minutes and removal of the supernatant; and washing the thawed platelets in step (h-1) comprises resuspending platelets in wash buffer, centrifugation at 1000×g for 15 minutes and removal of supernatant. In another aspect, the wash buffer further comprises about 50 mM trehalose. In another aspect, isolating the thawed platelets in step (h) comprises centrifugation at 1000×g for 5 minutes and removal of the supernatant; and washing the thawed platelets in step (h-1) comprises resuspending platelets in wash buffer, centrifugation at 1000×g for 5 minutes and removal of supernatant. In another aspect, the period of time in step (f) is from 1 day to about 5 years. Another embodiment described herein is recovered cryopreserved platelets prepared by the methods described herein.

Another embodiment described herein is a method for activation of recovered platelets after cryopreservation, the method comprising: (a) recovering platelets after cryopreservation according to the methods described herein; (b) treating the recovered platelets with platelet factor 4 (PF4; SEQ ID NO: 2); and (c) incubating the PF4-treated platelets with a plasma, serum, or blood sample from a subject diagnosed with heparin induced thrombocytopenia (HIT). In one aspect, treating the recovered platelets with PF4 comprises adding PF4 to the platelets at a concentration of about 150 μg/mL and incubating for about 20 minutes at room temperature. In another aspect, activation of the platelets is assessed by measuring platelet granule content released after incubation of the PF4-treated platelets with the plasma, serum, or blood sample from the HIT subject. In another aspect, the platelet granule content is thrombospondin-1. Another embodiment described herein is activated recovered cryopreserved platelets prepared by the methods described herein.

Another embodiment described herein is a method for activation of recovered platelets after cryopreservation, the method comprising: (a) recovering platelets after cryopreservation according to the methods described herein; and (b) treating the recovered platelets with Thrombin Receptor Activating Peptide (TRAP; amino acid sequence: SFFLRN; SEQ ID NO: 1). In one aspect, treating the recovered platelets with TRAP comprises adding TRAP to the platelets a concentration of about 25 μg/mL and incubating for about 30 minutes at room temperature. In another aspect, activation of the platelets is assessed by measuring platelet granule content released after treating the recovered platelets with TRAP. In another aspect, the platelet granule content is thrombospondin-1. Another embodiment described herein is activated recovered cryopreserved platelets prepared by the methods described herein.

Another embodiment described herein is a method for activation of recovered platelets after cryopreservation, the method comprising: (a) recovering platelets after cryopreservation according to the methods described herein; (b) treating the recovered platelets with heparin; and (c) incubating the heparin-treated platelets with a plasma, serum, or blood sample from a subject diagnosed with heparin induced thrombocytopenia (HIT). In one aspect, treating the recovered platelets with heparin comprises adding heparin to the platelets at a concentration of about 0.1-5 U/mL and incubating for about 20 minutes at room temperature. In another aspect, activation of the platelets is assessed by measuring platelet granule content released after incubation of the heparin-treated platelets with the plasma, serum, or blood sample from the HIT subject. In another aspect, the platelet granule content is thrombospondin-1. Another embodiment described herein is activated recovered cryopreserved platelets prepared by the methods described herein.

Another embodiment described herein is recovered cryopreserved platelets produced by the following method: (a) obtaining blood, platelet-rich plasma, or apheresis-derived platelets from a subject: (b) isolating platelets from the blood, platelet-rich plasma, or apheresis platelets; (c) suspending the platelets in a buffer solution comprising trehalose; (d) adding a stabilizing agent to the suspended platelets; (e) cooling the platelets to a temperature of about −80° C.; (f) storing the platelets at about −80° C. for a period of time; and (g) thawing the platelets at about 20-40° C.

Another embodiment described herein is activated recovered cryopreserved platelets prepared by the following method: (a) obtaining blood, platelet-rich plasma, or apheresis-derived platelets from a subject; (b) isolating platelets from the blood, platelet-rich plasma, or apheresis platelet; (c) suspending the platelets in a buffer solution comprising trehalose; (d) adding a stabilizing agent to the suspended platelets; (e) cooling the platelets to a temperature of about −80° C.; (f) storing the platelets at about −80° C. for a period of time; (g) thawing the platelets at about 20-40° C.; (h) isolating the thawed platelets; (i) suspending the isolated thawed platelets in an activation buffer comprising BSA at about 1% by mass; (j) treating the recovered platelets with heparin or PF4; and (k) incubating the heparin- or PF4-treated platelets with a plasma, serum, or blood sample from a subject diagnosed with heparin induced thrombocytopenia (HIT).

Another embodiment described herein is activated recovered cryopreserved platelets prepared by the following method: (a) obtaining blood, platelet-rich plasma, or apheresis-derived platelets from a subject; (b) isolating platelets from the blood, platelet-rich plasma, or apheresis platelet; (c) suspending the platelets in a buffer solution comprising trehalose; (d) adding a stabilizing agent to the suspended platelets; (e) cooling the platelets to a temperature of about −80° C.; (f) storing the platelets at about −80° C. for a period of time; (g) thawing the platelets at about 20-40° C.; (h) isolating the thawed platelets; (i) suspending the isolated thawed platelets in an activation buffer comprising BSA at about 1% by mass; and (j) treating the recovered platelets with Thrombin Receptor Activating Peptide (TRAP; amino acid sequence: SFFLRN; SEQ ID NO: 1).

Another embodiment described herein is a kit for the detection of heparin-induced thrombocytopenia (HIT) antibodies in blood, plasma or serum, the kit comprising two or more of: (a) cryopreserved platelets; (b) one or more of platelet factor 4 (PF4; SEQ ID NO: 2) or heparin; (c) one or more reagents for detecting platelet activation; (d) optionally, buffers and receptacles; and (e) optionally, one or more of packaging or instruction for use. In one aspect, the one or more reagents for detecting platelet activation comprise reagents for the detection of thrombospondin-1. In another aspect, the kit further comprises one or more negative control samples negative for platelet-activating pathogenic HIT antibodies; and one or more positive control samples positive for platelet-activating pathogenic HIT antibodies.

Another embodiment described herein is a method for treating or prophylaxis of hemorrhage in a subject in need thereof, the method comprising: (a) obtaining cryopreserved platelets; (b) thawing the cryopreserved platelets; (c) contacting the subject with the thawed cryopreserved platelets. In one aspect, steps (a)-(b) comprise: (i) obtaining blood, platelet-rich plasma, or apheresis-derived platelets from a second subject; (ii) isolating platelets from the blood, platelet-rich plasma, or apheresis platelets; (iii) suspending the platelets in a buffer solution comprising trehalose; (iv) incubating trehalose-treated platelets at 37° C. for about 2 hours; (v) adding a stabilizing agent to the suspended platelets; (vi) cooling the platelets to a temperature of about −80° C.; (vii) storing the platelets at about −80° C. for a period of time; and (viii) thawing the platelets at about 20-40° C. In another aspect, steps (a)-(b) comprise: (i) obtaining platelet-rich plasma or apheresis-derived platelets from a second subject; (ii) adding trehalose to the platelet rich plasma or apheresis-derived platelets; (iii) incubating trehalose-treated platelets at 37° C. for about 2 hours; (iv) cooling the platelets to a temperature of about −80° C.; (v) storing the platelets at about −80° C. for a period of time; and (vi) thawing the platelets at about 20-40° C. In another aspect, the contacting in step (c) comprises applying the thawed cryopreserved platelets to an injury situs or parenterally administering the thawed cryopreserved platelets to the subject.

Another embodiment described herein is the use of cryopreserved platelets as a reagent, research tool, or as a medicament for treating a subject in need thereof.

Another embodiment described herein is the use of thawed cryopreserved platelets as a reagent, research tool, or as a medicament for treating a subject in need thereof.

Another embodiment described herein is the use of activated, thawed cryopreserved platelets as a reagent, research tool, or as a medicament for treating a subject in need thereof.

### DESCRIPTION OF THE DRAWINGS

FIG. 1 shows exemplary process steps for cryopreservation and recovery of platelets as described herein.

FIG. 2 shows 20 lots of platelets from platelet-rich plasma units obtained from 20 different whole blood donors that were cryopreserved and recovered by washing. Platelets were treated with platelet factor 4 (PF4) and tested for reactivity against 3 HIT (closed circles) and 3 normal blood samples (open circles). Supernatant from the activation reaction was then assayed for thrombospondin-1 (TSP1) concentration, as a marker of platelet activation. The x-axis shows 20 different platelet lots, the y-axis depicts the amount of TSP1 released from the frozen platelets in μg/mL. Data are the mean of triplicate determinations.

FIG. 3 shows 7 lots of platelets that were cryopreserved and recovered by washing either after one week or less of storage at −80° C. (baseline, B) or after 4 weeks or more of storage at −80° C. (4 W). After recovery by washing, platelets were treated with PF4 and tested for reactivity against 3 HIT (closed circles) and 3 normal blood samples (open circles). Supernatant from the activation reaction was then assayed for TSP1 concentration as a marker of platelet activation. The x-axis depicts the 7 different platelet lots separated by vertical lines with both the baseline and 4-week timepoints, and the y-axis depicts the amount of TSP1 released from the frozen platelets in μg/mL. Data are the mean of triplicate determinations.

FIG. 4 shows platelets that were cryopreserved and recovered by washing after 14 weeks of storage at −80° C. After recovery, platelets were treated with PF4 and tested for reactivity against HIT (black) or a normal blood sample (white). Supernatant from cryopreserved platelets was then assayed for TSP1 concentration as a marker of platelet activation. The y-axis depicts the amount of TSP1 released from the frozen platelets in μg/mL. Data are the mean of triplicate determinations +1 standard deviation of the mean.

FIG. 5 shows platelets from a single donor that were cryopreserved and recovered by thawing and washing (1 Wash), thawing, and volume depleting (Volume Depletion), or thawing only (No Wash). Platelets were then treated with PF4. HIT (black) or normal blood sample (white) was added to the PF4-treated platelets. Platelet supernatant was assayed for TSP1 concentration as a marker of platelet activation. The y-axis depicts the amount of TSP1 released from the frozen platelets in μg/mL. Data shown is the mean of triplicate determinations +1 standard deviation of the mean.

FIG. 6 shows platelets from a single donor that were cryopreserved and recovered by thawing and washing (“1 Wash”), thawing and volume depleting (“Volume Depletion”), or thawing only (“No Wash”). TRAP (black) or buffer (white) was added to recovered platelets. Platelet supernatant was assayed for TSP1 concentration as a marker of platelet activation. The y-axis depicts the amount of TSP1 released from the frozen platelets in μg/mL. Data are the mean of triplicate determinations +1 standard deviation of the mean.

FIG. 7 shows platelets cryopreserved and recovered by washing, then treated with unfractionated heparin at concentrations of 0, 0.1, 0.3, 0.5, and 1 U/mL. HIT (black) or normal blood sample (white) was then added to heparin-treated platelets, and the supernatant was assayed for TSP1 concentration as a measure of platelet activation. The x-axis depicts heparin concentration, and y-axis depicts the amount of TSP1 released from the frozen platelets in μg/mL. Data are the mean of triplicate determinations +1 standard deviation of the mean.

FIG. 8 shows platelets that were cryopreserved with platelet poor plasma, recovered by washing and treated with PF4. HIT (black) or normal blood sample (white) was then added to PF4-treated platelets, and supernatant was assayed for TSP1 concentration as a measure of platelet activation. The y-axis depicts the amount of TSP1 released from the frozen platelets in μg/mL. Data are the mean of triplicate determinations +1 standard deviation of the mean.

FIG. 9 shows platelets that were cryopreserved in the presence of platelet poor plasma and recovered by washing. TRAP (black) or buffer (white) was then added and supernatant was assayed for TSP1 concentration as a measure of platelet activation. The y-axis depicts the amount of TSP1 released from the frozen platelets in μg/mL. Data are the mean of triplicate determinations +1 standard deviation of the mean.

FIG. 10 shows platelets from a single donor that were frozen and recovered by washing (black) or lyophilized and reconstituted (white), and then treated with PF4. HIT (left) or normal blood sample (right) was added to PF4-treated platelets, and supernatant was assayed for TSP1 concentration. The y-axis depicts the amount of TSP1 released from the frozen platelets in μg/mL. Data are the mean of triplicate determinations +1 standard deviation of the mean.

FIG. 11 shows platelets from a single donor that were frozen and recovered by washing (black) or lyophilized and reconstituted (white). TRAP (left) or buffer (right) was added to platelets, and supernatant was assayed for TSP1 concentration. The y-axis depicts the amount of TSP1 released from the frozen platelets in μg/mL. Data are the mean of triplicate determinations +1 standard deviation of the mean.

FIG. 12 shows platelets that were frozen and recovered by washing utilizing 15-minute centrifugation with no added trehalose in the resuspension buffers (left), or 5-minute centrifugation in the presence of 50 mM trehalose (right). HIT serum (black) or normal serum (white) was added to platelets, and supernatant was assayed for TSP1 concentration. The y-axis depicts the amount of TSP1 released from the frozen platelets in μg/mL. Data is the mean of triplicate determinations +1 standard deviation of the mean.

FIG. 13 shows stabilized platelets that were treated with an agonist in the presence and absence of candidate drug that antagonizes agonist action. Such stabilized platelets can replace the requirement for fresh platelets in these studies.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 1.5-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used interchangeably. A “reference” or “control” level may be a predetermined value or range, which is employed as a baseline or benchmark against which to assess a measured result. “Control” also refers to control experiments or control cells.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

The compositions described herein may be administered parenterally, topically, rectally, nasally, buccally, vaginally, or via an implanted reservoir. The term “parenterally” or “parenteral administration” as used herein includes, intravenous, intraarterial, subcutaneous, intramuscular, intra-articular, intra-synovial, intrasternal, intrathecal, intrahepatic, intralesional, intracranial injection, or via systemic infusion. In some embodiments, the compositions described herein are administered intravenously, intraarterially, or by infusion.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human. In one aspect, the subject is suspected of having pathogenic antibodies or platelet-activating antibodies. A “subject suspected of having platelet-activating antibodies” or “test subject” as used herein refers to a subject exhibiting clinical findings indicative of pathogenic antibodies, including, for example, a below-normal platelet count, a decrease in platelet count, enlargement or extension of a previously diagnosed blood clot, or the development of a new blood clot elsewhere in the body. Symptoms indicative of pathogenic antibodies include shortness of breath, stroke, limb gangrene, skin necrosis, myocardial infarction, or chest pain. In one embodiment the platelet-activating antibody is an antibody capable of causing HIT. In another embodiment, the platelet-activating antibodies comprise platelet-activating heparin-induced thrombocytopenia antibodies. A “normal subject” refers to a subject not suffering from any aliment or not suspected of having platelet-activating antibodies.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, “identifying” or “diagnosing” refers to classifying a subject as having a pathology or a symptom, determining a severity of the pathology (grade or stage), monitoring pathology progression, or forecasting an outcome of a pathology or prospects of recovery. In one embodiment, a subject is identified or diagnosed as having platelet-activating antibodies in its blood. In one aspect, the platelet-activating antibodies comprise platelet-activating heparin-induced thrombocytopenia antibodies. The identification or detection of platelet-activating heparin-induced thrombocytopenia antibodies is indicative or diagnostic of the subject having heparin-induced thrombocytopenia (HIT).

As used herein, “heparin-induced thrombocytopenia” or “HIT” refers to an adverse reaction to heparin or thrombotic thrombocytopenia reactions occurring in the absence of heparin exposure such as Spontaneous HIT and “Vaccine-induced immune thrombotic thrombocytopenia” or “VITT”, in which affected subjects produce platelet-activating antibodies that bind molecules such as PF4, IL-8 and NAP-2, either in a complex with heparin, in complex with platelet membrane components, or alone resulting in a prothrombotic and thrombocytopenic condition that can be life-threatening.

As used herein, “heparin-induced” refers to antibodies that result from exposure to heparin or those that recognize blood factors complexed with heparin or platelet membrane components as in spontaneous HIT or VITT, as indicative of HIT.

As used herein, “heparin-like compound” refers to a compound with a high negative charge such as a polyanion, a heparin derivative, a chemically modified heparin, a heparin-like glycosaminoglycan molecule, a proteoglycan containing multiple heparin or heparin-like glycosaminoglycans, lower-molecular-weight heparin, a synthetic glycosaminoglycan comprising at least 10 saccharide units, or a synthetic heparin-like glycosaminoglycan any of which could be connected directly or through a spacer/linker molecule to a core molecule.

“Platelets,” also known as “thrombocytes,” as used herein refer to the anucleate fragments of megakaryocytes involved in blood coagulation, hemostasis, and blood thrombus formation. Human platelets are routinely isolated through a variety of methods including platelet apheresis, plateletpheresis, gel filtration, or differential centrifugation. Isolated platelets would be suitable, however, platelets from other sources, including washed platelets, unwashed platelets, platelet rich plasma or purified platelets, could also be used. In one embodiment, the platelets have been stabilized for storage by one or more of cooling, freezing, chemical storage, or lyophilization.

“Platelet activation” as used herein refers to the response of platelets when platelets encounter a “platelet activator” molecule that triggers activation, such as platelet activating antibodies, Thrombin Receptor Activating Peptide (TRAP, SEQ ID NO: 1), adenosine diphosphate (ADP), arachidonic acid, epinephrine, collagen, thrombin, thromboxane A2 (TxA2), thromboxane A2 (TXA2) mimetic U46619, calcium ionophore A23187, ristocetin, rhodocytin, among others. Platelet activation results in various changes to the platelets, including, for example, changes in markers associated with platelet activation, exocytosis of the dense granules and alpha granules, activation of the membrane enzyme phospholipase A2, changes in shape, aggregation, agglutination, changes in membrane potential, changes in integrin conformation, inter alia. Upon activation, platelets release granule contents including: adenosine triphosphate (ATP), adenosine diphosphate (ADP), 5-hydroxytryptamine (serotonin), thrombospondin-1, fibrinogen, CXCL12, thromboxanes, platelet factor 4, among other metabolites and proteins, which may be assessed by using different methodologies such as immunological assay, high-performance liquid chromatography (HPLC), fluorescence microscopy, or flow cytometry. Upon activation, platelets have a change in surface expression various markers including of P-Selectins, CD34, CD41, CD61, phosphatidyl serine, among others which may be assessed by using different methodologies such as immunological assay, high-performance liquid chromatography (HPLC), fluorescence microscopy, or flow cytometry.

An “effective amount” as used herein refers to an amount of a compound that is sufficient to affect the desired outcome. In one embodiment, platelets are activated by administering an effective amount of one or more platelet activators as described herein.

Platelet activation levels may be measured using any method known in the art, such as, for instance, measuring levels of a marker found on or released from activated platelets in a reaction of normal donor platelets with a blood sample from a subject known to have platelet-activating antibodies or HIT (e.g., positive control sample) and with a normal subject's blood sample (e.g., a negative control sample). Any marker known to be found on or released from activated platelets may be used to measure platelet activation, including, for example, one or more CD markers found on activated platelets (e.g., CD9, CD31, CD36, CD41, CD42, CD49b, CD61, CD62P, CD63, CD107, their isoforms, or any marker present on the outside of platelets), including the marker CD62P, also known as p-selectin. In addition, platelet activation can be measured by measuring any increased binding of immunoglobulin, phosphatidyl serine expression, platelet aggregation, intracellular levels of ionized calcium, changes in integrin conformation, release of platelet granule contents, changes in platelet membrane potential or platelet impedance, levels of Fc gamma receptor 2 cleavage fragments, or shape change of platelets.

“Increase in platelet activation” as used herein refers to an increase in platelet activation that is significantly different than that of the baseline platelet activation in a normal subject's blood sample or in comparison to known values or historical data obtained using the assays described herein. An increase in platelet activation is indicative of the patient having platelet-activating antibodies, such as antibodies that cause HIT. In one embodiment, the increase in platelet activation is at least two or three times the amount of baseline platelet activation in normal subject blood samples or historical data obtained for normal subjects using the methods described herein.

A “sample” as used herein refers to a specimen or culture obtained from a subject. Biological samples can be obtained from subjects and encompass fluids, solids, tissues, and gases. In one embodiment, the subject's sample is a blood sample. Blood samples include whole blood, plasma, serum, blood products such as platelet-rich plasma, or fractionated blood components, such as one of the Cohn fractions I-IV, or an antibody fraction.

“Instructions for use” as used herein refers to a publication, diagram, or any other medium of expression which is used to provide instructions or steps for performing the methods described herein. The instructions for use can be provided in printed form, affixed to a container which contains the kit materials, shipped together with the kit, or provided at an internet site.

One embodiment described herein is a method for cryopreserving platelets that maintains viability and activation potential, the method comprising: (a) obtaining blood or platelet-rich plasma, or apheresis-derived platelets from a subject; (b) isolating platelets from the blood, platelet-rich plasma, or apheresis platelets; (c) suspending the platelets in a suspension buffer comprising trehalose; (d) adding a stabilizing agent to the suspended platelets; (e) cooling the platelets to a temperature of about −80° C.; and (f) storing the platelets at about −80° C. In one aspect, the method maintains the viability and activation potential of the platelets for greater than or equal to 2 weeks. In another aspect, the suspension buffer comprises about 50 mM trehalose. In another aspect, the stabilizing agent is bovine serum albumin (BSA) at about 4% by mass. In another aspect, the platelets of step (c) are diluted with suspension buffer to a final concentration of about 1×10⁶ platelets/μL. In another aspect, the diluted platelets are incubated at about 37° C. for about 2 hours with periodic agitation. In another aspect, isolating the platelets comprises centrifugation of the blood, platelet-rich plasma, or apheresis platelets at 100×g for 15 minutes to generate a first pellet and a first supernatant; followed by centrifugation of the first supernatant at 1000×g for 15 minutes to generate a second pellet and a second supernatant, wherein the first supernatant is platelet-rich and the second supernatant is platelet-poor. In another aspect, the second supernatant is collected, trehalose is added to about 50 mM, and is used to dilute the platelets of step (c) to a concentration of about 1×10⁸ platelets/μL. In another aspect, cooling the platelets comprises decreasing the temperature at a rate of about 2-20° C. per minute. Another embodiment described herein is cryopreserved platelets prepared by the methods described herein.

Another embodiment described herein is a method for cryopreserving and recovering platelets from cryopreservation, the method comprising: (a) obtaining blood, platelet-rich plasma, or apheresis-derived platelets from a subject; (b) isolating platelets from the blood, platelet-rich plasma, or apheresis platelets; (c) suspending the platelets in a buffer solution comprising trehalose; (d) adding a stabilizing agent to the suspended platelets; (e) cooling the platelets to a temperature of about −80° C.; (f) storing the platelets at about −80° C. for a period of time; and (g) thawing the platelets at about 20-40° C. In one aspect, the suspension buffer comprises about 50 mM trehalose. In another aspect, the stabilizing agent is bovine serum albumin (BSA) at about 4% by mass. In another aspect, the platelets of step (c) are diluted with suspension buffer to a final concentration of about 1×10⁶ platelets/μL. In another aspect, the diluted platelets are incubated at about 37° C. for about 2 hours with periodic agitation. In another aspect, the method further comprises the steps: (h) isolating the thawed platelets; and (i) suspending the isolated thawed platelets in an activation buffer comprising BSA at about 1% by mass. In another aspect, isolating the thawed platelets comprises centrifugation at 1000×g for 15 minutes. In another aspect, the activation buffer further comprises about 50 mM trehalose. In another aspect, isolating the thawed platelets comprises centrifugation at 1000×g for 5 minutes. In another aspect, after step (h) and prior to step (i), the following step is performed: (h-1) washing the thawed platelets in a wash buffer. In another aspect, isolating the thawed platelets in step (h) comprises centrifugation at 1000×g for 15 minutes and removal of the supernatant; and washing the thawed platelets in step (h-1) comprises resuspending platelets in wash buffer, centrifugation at 1000×g for 15 minutes and removal of supernatant. In another aspect, the wash buffer further comprises about 50 mM trehalose. In another aspect, isolating the thawed platelets in step (h) comprises centrifugation at 1000×g for 5 minutes and removal of the supernatant; and washing the thawed platelets in step (h-1) comprises resuspending platelets in wash buffer, centrifugation at 1000×g for 5 minutes and removal of supernatant. In another aspect, the period of time in step (f) is from 1 day to about 5 years. Another embodiment described herein is recovered cryopreserved platelets prepared by the methods described herein.

Another embodiment described herein is a method for activation of recovered platelets after cryopreservation, the method comprising: (a) recovering platelets after cryopreservation according to the methods described herein; (b) treating the recovered platelets with platelet factor 4 (PF4; SEQ ID NO: 2); and (c) incubating the PF4-treated platelets with a plasma, serum, or blood sample from a subject diagnosed with heparin induced thrombocytopenia (HIT). In one aspect, treating the recovered platelets with PF4 comprises adding PF4 to the platelets at a concentration of about 150 μg/mL and incubating for about 20 minutes at room temperature. In another aspect, activation of the platelets is assessed by measuring platelet granule content released after incubation of the PF4-treated platelets with the plasma, serum, or blood sample from the HIT subject. In another aspect, the platelet granule content is thrombospondin-1. Another embodiment described herein is activated recovered cryopreserved platelets prepared by the methods described herein.

Another embodiment described herein is a method for activation of recovered platelets after cryopreservation, the method comprising: (a) recovering platelets after cryopreservation according to the methods described herein; and (b) treating the recovered platelets with Thrombin Receptor Activating Peptide (TRAP; amino acid sequence: SFFLRN; SEQ ID NO: 1). In one aspect, treating the recovered platelets with TRAP comprises adding TRAP to the platelets at a concentration of about 25 g/mL and incubating for about 30 minutes at room temperature. In another aspect, activation of the platelets is assessed by measuring platelet granule content released after treating the recovered platelets with TRAP. In another aspect, the platelet granule content is thrombospondin-1. Another embodiment described herein is activated recovered cryopreserved platelets prepared by the methods described herein.

Another embodiment described herein is a method for activation of recovered platelets after cryopreservation, the method comprising: (a) recovering platelets after cryopreservation according to the methods described herein; (b) treating the recovered platelets with heparin; and (c) incubating the heparin-treated platelets with a plasma, serum, or blood sample from a subject diagnosed with heparin induced thrombocytopenia (HIT). In one aspect, treating the recovered platelets with heparin comprises adding heparin to the platelets at a concentration of about 0.1-5 U/mL and incubating for about 20 minutes at room temperature. In another aspect, activation of the platelets is assessed by measuring platelet granule content released after incubation of the heparin-treated platelets with the plasma, serum, or blood sample from the HIT subject. In another aspect, the platelet granule content is thrombospondin-1. Another embodiment described herein is activated recovered cryopreserved platelets prepared by the methods described herein.

Another embodiment described herein is a method for cryopreserving platelets and recovering platelets from cryopreservation, the method comprising: (a) obtaining blood, platelet-rich plasma, or apheresis-derived platelets from a subject; (b) isolating platelets from the blood, platelet-rich plasma, or apheresis platelet; (c) suspending the platelets in a buffer solution comprising trehalose; (d) adding a stabilizing agent to the suspended platelets; (e) cooling the platelets to a temperature of about −80° C.; (f) storing the platelets at about −80° C. for a period of time; (g) thawing the platelets at about 20-40° C.; (h) isolating the thawed platelets; and (i) suspending the isolated thawed platelets in an activation buffer comprising BSA at about 1% by mass. In one aspect, the suspension buffer comprises about 50 mM trehalose. In another aspect, the stabilizing agent is bovine serum albumin (BSA) at about 4% by mass. In another aspect, the platelets of step (c) are diluted with suspension buffer to a final concentration of about 1×10⁶ platelets/μL. In another aspect, the diluted platelets are incubated at about 37° C. for about 2 hours with periodic agitation. In another aspect, isolating the thawed platelets comprises centrifugation at 1000×g for 15 minutes. In another aspect, the activation buffer further comprises about 50 mM trehalose. In another aspect, isolating the thawed platelets comprises centrifugation at 1000×g for 5 minutes. In another aspect, after step (h) and prior to step (i), the following step is performed: (h-1) washing the thawed platelets in a wash buffer. In another aspect, isolating the thawed platelets in step (h) comprises centrifugation at 1000×g for 15 minutes and removal of the supernatant; and washing the thawed platelets in step (h-1) comprises resuspending platelets in wash buffer, centrifugation at 1000×g for 15 minutes and removal of supernatant. In another aspect, the wash buffer further comprises about 50 mM trehalose. In another aspect, isolating the thawed platelets in step (h) comprises centrifugation at 1000×g for 5 minutes and removal of the supernatant; and washing the thawed platelets in step (h-1) comprises resuspending platelets in wash buffer, centrifugation at 1000×g for 5 minutes and removal of supernatant. In another aspect, the period of time in step (f) is from 1 day to about 5 years. Another embodiment described herein is recovered cryopreserved platelets prepared by the methods described herein.

Another embodiment described herein is a method for activation of recovered platelets after cryopreservation, the method comprising: (a) recovering platelets after cryopreservation according to the methods described herein; (b) treating the recovered platelets with platelet factor 4 (PF4; SEQ ID NO: 2); and (c) incubating the PF4-treated platelets with a plasma, serum, or blood sample from a subject diagnosed with heparin induced thrombocytopenia (HIT). In one aspect, treating the recovered platelets with PF4 comprises adding PF4 to the platelets at a concentration of about 150 μg/mL and incubating for about 20 minutes at room temperature. In another aspect, activation of the platelets is assessed by measuring platelet granule content released after incubation of the PF4-treated platelets with the plasma, serum, or blood sample from the HIT subject. In another aspect, the platelet granule content is thrombospondin-1. Another embodiment described herein is activated recovered cryopreserved platelets prepared by the methods described herein.

Another embodiment described herein is a method for activation of recovered platelets after cryopreservation, the method comprising: (a) recovering platelets after cryopreservation according to the methods described herein; and (b) treating the recovered platelets with Thrombin Receptor Activating Peptide (TRAP; amino acid sequence: SFFLRN; SEQ ID NO: 1). In one aspect, treating the recovered platelets with TRAP comprises adding TRAP to the platelets a concentration of about 25 μg/mL and incubating for about 30 minutes at room temperature. In another aspect, activation of the platelets is assessed by measuring platelet granule content released after treating the recovered platelets with TRAP. In another aspect, the platelet granule content is thrombospondin-1. Another embodiment described herein is activated recovered cryopreserved platelets prepared by the methods described herein.

Another embodiment described herein is a method for activation of recovered platelets after cryopreservation, the method comprising: (a) recovering platelets after cryopreservation according to the methods described herein; (b) treating the recovered platelets with heparin; and (c) incubating the heparin-treated platelets with a plasma, serum, or blood sample from a subject diagnosed with heparin induced thrombocytopenia (HIT). In one aspect, treating the recovered platelets with heparin comprises adding heparin to the platelets at a concentration of about 0.1-5 U/mL and incubating for about 20 minutes at room temperature. In another aspect, activation of the platelets is assessed by measuring platelet granule content released after incubation of the heparin-treated platelets with the plasma, serum, or blood sample from the HIT subject. In another aspect, the platelet granule content is thrombospondin-1. Another embodiment described herein is activated recovered cryopreserved platelets prepared by the methods described herein.

Another embodiment described herein is recovered cryopreserved platelets produced by the following method: (a) obtaining blood, platelet-rich plasma, or apheresis-derived platelets from a subject: (b) isolating platelets from the blood, platelet-rich plasma, or apheresis platelets; (c) suspending the platelets in a buffer solution comprising trehalose; (d) adding a stabilizing agent to the suspended platelets; (e) cooling the platelets to a temperature of about −80° C.; (f) storing the platelets at about −80° C. for a period of time; and (g) thawing the platelets at about 20-40° C.

Another embodiment described herein is activated recovered cryopreserved platelets prepared by the following method: (a) obtaining blood, platelet-rich plasma, or apheresis-derived platelets from a subject; (b) isolating platelets from the blood, platelet-rich plasma, or apheresis platelet; (c) suspending the platelets in a buffer solution comprising trehalose; (d) adding a stabilizing agent to the suspended platelets; (e) cooling the platelets to a temperature of about −80° C.; (f) storing the platelets at about −80° C. for a period of time; (g) thawing the platelets at about 20-40° C.; (h) isolating the thawed platelets; (i) suspending the isolated thawed platelets in an activation buffer comprising BSA at about 1% by mass; (j) treating the recovered platelets with heparin or PF4; and (k) incubating the heparin- or PF4-treated platelets with a plasma, serum, or blood sample from a subject diagnosed with heparin induced thrombocytopenia (HIT).

Another embodiment described herein is activated recovered cryopreserved platelets prepared by the following method: (a) obtaining blood, platelet-rich plasma, or apheresis-derived platelets from a subject; (b) isolating platelets from the blood, platelet-rich plasma, or apheresis platelet; (c) suspending the platelets in a buffer solution comprising trehalose; (d) adding a stabilizing agent to the suspended platelets; (e) cooling the platelets to a temperature of about −80° C.; (f) storing the platelets at about −80° C. for a period of time; (g) thawing the platelets at about 20-40° C.; (h) isolating the thawed platelets; (i) suspending the isolated thawed platelets in an activation buffer comprising BSA at about 1% by mass; and (j) treating the recovered platelets with Thrombin Receptor Activating Peptide (TRAP; amino acid sequence: SFFLRN; SEQ ID NO: 1).

Another embodiment described herein is a kit for the detection of heparin-induced thrombocytopenia (HIT) antibodies in blood, plasma or serum, the kit comprising two or more of: (a) cryopreserved platelets; (b) one or more of platelet factor 4 (PF4; SEQ ID NO: 2) or heparin; (c) one or more reagents for detecting platelet activation; (d) optionally, buffers and receptacles; and (e) optionally, one or more of packaging or instruction for use. In one aspect, the one or more reagents for detecting platelet activation comprise reagents for the detection of thrombospondin-1. In another aspect, the kit further comprises one or more negative control samples negative for platelet-activating pathogenic HIT antibodies; and one or more positive control samples positive for platelet-activating pathogenic HIT antibodies.

Another embodiment described herein is a method for treating or prophylaxis of hemorrhage in a subject in need thereof, the method comprising: (a) obtaining cryopreserved platelets; (b) thawing the cryopreserved platelets; (c) contacting the subject with the thawed cryopreserved platelets. In one aspect, steps (a)-(b) comprise: (i) obtaining blood, platelet-rich plasma, or apheresis-derived platelets from a second subject; (ii) isolating platelets from the blood, platelet-rich plasma, or apheresis platelets; (iii) suspending the platelets in a buffer solution comprising trehalose; (iv) incubating trehalose-treated platelets at 37° C. for about 2 hours; (v) adding a stabilizing agent to the suspended platelets; (vi) cooling the platelets to a temperature of about −80° C.; (vii) storing the platelets at about −80° C. for a period of time; and (viii) thawing the platelets at about 20-40° C. In another aspect, steps (a)-(b) comprise: (i) obtaining platelet-rich plasma or apheresis-derived platelets from a second subject; (ii) adding trehalose to the platelet rich plasma or apheresis-derived platelets; (iii) incubating trehalose-treated platelets at 37° C. for about 2 hours; (iv) cooling the platelets to a temperature of about −80° C.; (v) storing the platelets at about −80° C. for a period of time; and (vi) thawing the platelets at about 20-40° C. In another aspect, the contacting in step (c) comprises applying the thawed cryopreserved platelets to an injury situs or parenterally administering the thawed cryopreserved platelets to the subject.

Another embodiment described herein is the use of cryopreserved platelets as a reagent, research tool, or as a medicament for treating a subject in need thereof.

Another embodiment described herein is the use of thawed cryopreserved platelets as a reagent, research tool, or as a medicament for treating a subject in need thereof.

Another embodiment described herein is the use of activated, thawed cryopreserved platelets as a reagent, research tool, or as a medicament for treating a subject in need thereof.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method for cryopreserving platelets that maintains viability and activation potential, the method comprising:
   (a) obtaining blood or platelet-rich plasma, or apheresis-derived platelets from a subject;
   (b) isolating platelets from the blood, platelet-rich plasma, or apheresis platelets;
   (c) suspending the platelets in a suspension buffer comprising trehalose;
   (d) adding a stabilizing agent to the suspended platelets;
   (e) cooling the platelets to a temperature of about −80° C.; and
   (f) storing the platelets at about −80° C.
Clause 2. The method of clause 1, wherein the method maintains the viability and activation potential of the platelets for greater than or equal to 2 weeks.
Clause 3. The method of clause 1 or 2, wherein the suspension buffer comprises about 50 mM trehalose.
Clause 4. The method of any one of clauses 1-3, wherein the stabilizing agent is bovine serum albumin (BSA) at about 4% by mass.
Clause 5. The method of any one of clauses 1-4, wherein the platelets of step (c) are diluted with suspension buffer to a final concentration of about 1×10⁶ platelets/μL.
Clause 6. The method of any one of clauses 1-5, wherein the diluted platelets are incubated at about 37° C. for about 2 hours with periodic agitation.
Clause 7. The method of any one of clauses 1-6, wherein isolating the platelets comprises centrifugation of the blood, platelet-rich plasma, or apheresis platelets at 100×g for 15 minutes to generate a first pellet and a first supernatant; followed by centrifugation of the first supernatant at 1000×g for 15 minutes to generate a second pellet and a second supernatant, wherein the first supernatant is platelet-rich and the second supernatant is platelet-poor.
Clause 8. The method of any one of clauses 1-7, wherein the second supernatant is collected, trehalose is added to about 50 mM, and is used to dilute the platelets of step (c) to a concentration of about 1×10⁶ platelets/μL.
Clause 9. The method of any one of clauses 1-8, wherein cooling the platelets comprises decreasing the temperature at a rate of about 2-20° C. per minute.
Clause 10. Cryopreserved platelets prepared by the method of any one of clauses 1-9.
Clause 11. A method for cryopreserving and recovering platelets from cryopreservation, the method comprising:
   (a) obtaining blood, platelet-rich plasma, or apheresis-derived platelets from a subject;
   (b) isolating platelets from the blood, platelet-rich plasma, or apheresis platelets;
   (c) suspending the platelets in a buffer solution comprising trehalose;
   (d) adding a stabilizing agent to the suspended platelets;
   (e) cooling the platelets to a temperature of about −80° C.;
   (f) storing the platelets at about −80° C. for a period of time; and
   (g) thawing the platelets at about 20-40° C.
Clause 12. The method of clause 11, wherein the suspension buffer comprises about 50 mM trehalose.
Clause 13. The method of clause 11 or 12, wherein the stabilizing agent is bovine serum albumin (BSA) at about 4% by mass.
Clause 14. The method of any one of clauses 11-13, wherein the platelets of step (c) are diluted with suspension buffer to a final concentration of about 1×10⁶ platelets/μL.
Clause 15. The method of any one of clauses 11-14, wherein the diluted platelets are incubated at about 37° C. for about 2 hours with periodic agitation.
Clause 16. The method of any one of clauses 11-15, further comprising the steps:
   (h) isolating the thawed platelets; and
   (i) suspending the isolated thawed platelets in an activation buffer comprising BSA at about 1% by mass.
Clause 17. The any one of clauses 11-16, wherein isolating the thawed platelets comprises centrifugation at 1000×g for 15 minutes.
Clause 18. The method of any one of clauses 11-17, wherein the activation buffer further comprises about 50 mM trehalose.
Clause 19. The method of any one of clauses 11-18, wherein isolating the thawed platelets comprises centrifugation at 1000×g for 5 minutes.
Clause 20. The method of any one of clauses 11-19, wherein after step (h) and prior to step (i), the following step is performed:
   (h-1) washing the thawed platelets in a wash buffer.
Clause 21. The method of clause 20, wherein isolating the thawed platelets in step (h) comprises centrifugation at 1000×g for 15 minutes and removal of the supernatant; and washing the thawed platelets in step (h-1) comprises resuspending platelets in wash buffer, centrifugation at 1000×g for 15 minutes and removal of supernatant.
Clause 22. The method of any one of clauses 11-21, wherein the wash buffer further comprises about 50 mM trehalose.
Clause 23. The method of any one of clauses 11-22, wherein isolating the thawed platelets in step (h) comprises centrifugation at 1000×g for 5 minutes and removal of the supernatant; and washing the thawed platelets in step (h-1) comprises resuspending platelets in wash buffer, centrifugation at 1000×g for 5 minutes and removal of supernatant.
Clause 24. The method of any one of clauses 11-24, wherein the period of time in step (f) is from 1 day to about 5 years.
Clause 25. Recovered cryopreserved platelets prepared by the method of any one of clauses 11-24.
Clause 26. A method for activation of recovered platelets after cryopreservation, the method comprising:
   (a) recovering platelets after cryopreservation according to the method of any one of clauses 11-24;
   (b) treating the recovered platelets with platelet factor 4 (PF4; SEQ ID NO: 2); and
   (c) incubating the PF4-treated platelets with a plasma, serum, or blood sample from a subject diagnosed with heparin induced thrombocytopenia (HIT).
Clause 27. The method of clause 26, wherein treating the recovered platelets with PF4 comprises adding PF4 to the platelets at a concentration of about 150 μg/mL and incubating for about 20 minutes at room temperature.
Clause 28. The method of clause 26 or 27, wherein activation of the platelets is assessed by measuring platelet granule content released after incubation of the PF4-treated platelets with the plasma, serum, or blood sample from the HIT subject.
Clause 29. The method of any one of clauses 26-28, wherein the platelet granule content is thrombospondin-1.
Clause 30. Activated recovered cryopreserved platelets prepared by the method of any one of clauses 26-29.
Clause 31. A method for activation of recovered platelets after cryopreservation, the method comprising:
   (a) recovering platelets after cryopreservation according to the method of any one of clauses 11-24; and
   (b) treating the recovered platelets with Thrombin Receptor Activating Peptide (TRAP; amino acid sequence: SFFLRN; SEQ ID NO: 1).
Clause 32. The method of clause 31, wherein treating the recovered platelets with TRAP comprises adding TRAP to the platelets at a concentration of about 25 μg/mL and incubating for about 30 minutes at room temperature.
Clause 33. The method of clause 31 or 32, wherein activation of the platelets is assessed by measuring platelet granule content released after treating the recovered platelets with TRAP.
Clause 34. The method of any one of clauses 31-33, wherein the platelet granule content is thrombospondin-1.
Clause 35. Activated recovered cryopreserved platelets prepared by the method of any one of clauses 31-34.
Clause 36. A method for activation of recovered platelets after cryopreservation, the method comprising:
   (a) recovering platelets after cryopreservation according to the method of any one of clauses 11-24;
   (b) treating the recovered platelets with heparin; and
   (c) incubating the heparin-treated platelets with a plasma, serum, or blood sample from a subject diagnosed with heparin induced thrombocytopenia (HIT).
Clause 37. The method of clause 36, wherein treating the recovered platelets with heparin comprises adding heparin to the platelets at a concentration of about 0.1-5 U/mL and incubating for about 20 minutes at room temperature.
Clause 38. The method of clause 36 or 37, wherein activation of the platelets is assessed by measuring platelet granule content released after incubation of the heparin-treated platelets with the plasma, serum, or blood sample from the HIT subject.
Clause 39. The method of any one of clauses 36-38, wherein the platelet granule content is thrombospondin-1.
Clause 40. Activated recovered cryopreserved platelets prepared by the method of one of clauses 36-39.
Clause 41. A method for cryopreserving platelets and recovering platelets from cryopreservation, the method comprising:
   (a) obtaining blood, platelet-rich plasma, or apheresis-derived platelets from a subject;
   (b) isolating platelets from the blood, platelet-rich plasma, or apheresis platelet;
   (c) suspending the platelets in a buffer solution comprising trehalose;
   (d) adding a stabilizing agent to the suspended platelets;
   (e) cooling the platelets to a temperature of about −80° C.;
   (f) storing the platelets at about −80° C. for a period of time;
   (g) thawing the platelets at about 20-40° C.;
   (h) isolating the thawed platelets; and
   (i) suspending the isolated thawed platelets in an activation buffer comprising BSA at about 1% by mass.
Clause 42. The method of clause 41, wherein the suspension buffer comprises about 50 mM trehalose.
Clause 43. The method of clause 41 or 42, wherein the stabilizing agent is bovine serum albumin (BSA) at about 4% by mass.
Clause 44. The method of any one of clauses 41-43, wherein the platelets of step (c) are diluted with suspension buffer to a final concentration of about 1×10⁶ platelets/μL.
Clause 45. The method of any one of clauses 41-44, wherein the diluted platelets are incubated at about 37° C. for about 2 hours with periodic agitation.
Clause 46. The method of any one of clauses 41-45, wherein isolating the thawed platelets comprises centrifugation at 1000×g for 15 minutes.
Clause 47. The method of any one of clauses 41-46, wherein the activation buffer further comprises about 50 mM trehalose.
Clause 48. The method of any one of clauses 41-47, wherein isolating the thawed platelets comprises centrifugation at 1000×g for 5 minutes.
Clause 49. The method of clause any one of clauses 41-48, wherein after step (h) and prior to step (i), the following step is performed:
   (h-1) washing the thawed platelets in a wash buffer.
Clause 50. The method of any one of clauses 41-49, wherein isolating the thawed platelets in step (h) comprises centrifugation at 1000×g for 15 minutes and removal of the supernatant; and washing the thawed platelets in step (h-1) comprises resuspending platelets in wash buffer, centrifugation at 1000×g for 15 minutes and removal of supernatant.
Clause 51. The method of any one of clauses 41-50, wherein the wash buffer further comprises about 50 mM trehalose.
Clause 52. The method of any one of clauses 41-51, wherein isolating the thawed platelets in step (h) comprises centrifugation at 1000×g for 5 minutes and removal of the supernatant; and washing the thawed platelets in step (h-1) comprises resuspending platelets in wash buffer, centrifugation at 1000×g for 5 minutes and removal of supernatant.
Clause 53. The method of any one of clauses 41-52, wherein the period of time in step (f) is from 1 day to about 5 years.
Clause 54. Recovered cryopreserved platelets prepared by the method of any one of clauses 41-53.
Clause 55. A method for activation of recovered platelets after cryopreservation, the method comprising:
   (a) recovering platelets after cryopreservation according to the method of any one of clauses 41-53;
   (b) treating the recovered platelets with platelet factor 4 (PF4; SEQ ID NO: 2); and
   (c) incubating the PF4-treated platelets with a plasma, serum, or blood sample from a subject diagnosed with heparin induced thrombocytopenia (HIT).
Clause 56. The method of clause 55, wherein treating the recovered platelets with PF4 comprises adding PF4 to the platelets at a concentration of about 150 μg/mL and incubating for about 20 minutes at room temperature.
Clause 57. The method of clause 55 or 56, wherein activation of the platelets is assessed by measuring platelet granule content released after incubation of the PF4-treated platelets with the plasma, serum, or blood sample from the HIT subject.
Clause 58. The method of any one of clauses 55-57, wherein the platelet granule content is thrombospondin-1.
Clause 59. Activated recovered cryopreserved platelets prepared by the method of any one of clauses 55-58.
Clause 60. A method for activation of recovered platelets after cryopreservation, the method comprising:
   (a) recovering platelets after cryopreservation according to the method of any one of clauses 41-53; and
   (b) treating the recovered platelets with Thrombin Receptor Activating Peptide (TRAP; amino acid sequence: SFFLRN; SEQ ID NO: 1).
Clause 61. The method of clause 60, wherein treating the recovered platelets with TRAP comprises adding TRAP to the platelets a concentration of about 25 μg/mL and incubating for about 30 minutes at room temperature.
Clause 62. The method of clause 60 or 61, wherein activation of the platelets is assessed by measuring platelet granule content released after treating the recovered platelets with TRAP.
Clause 63. The method of any one of clauses 60-62, wherein the platelet granule content is thrombospondin-1.
Clause 64. Activated recovered cryopreserved platelets prepared by the method of any one of clauses 60-62.
Clause 65. A method for activation of recovered platelets after cryopreservation, the method comprising:
   (a) recovering platelets after cryopreservation according to the method of any one of clauses 41-53;
   (b) treating the recovered platelets with heparin; and
   (c) incubating the heparin-treated platelets with a plasma, serum, or blood sample from a subject diagnosed with heparin induced thrombocytopenia (HIT).
Clause 66. The method of clause 65, wherein treating the recovered platelets with heparin comprises adding heparin to the platelets at a concentration of about 0.1-5 U/mL and incubating for about 20 minutes at room temperature.
Clause 67. The method of clause 65 or 66, wherein activation of the platelets is assessed by measuring platelet granule content released after incubation of the heparin-treated platelets with the plasma, serum, or blood sample from the HIT subject.
Clause 68. The method of any one of clauses 65-67, wherein the platelet granule content is thrombospondin-1.
Clause 69. Activated recovered cryopreserved platelets prepared by the method of any one of clauses 65-68.
Clause 70. Recovered cryopreserved platelets produced by the following method:
   (a) obtaining blood, platelet-rich plasma, or apheresis-derived platelets from a subject;
   (b) isolating platelets from the blood, platelet-rich plasma, or apheresis platelets;
   (c) suspending the platelets in a buffer solution comprising trehalose;
   (d) adding a stabilizing agent to the suspended platelets;
   (e) cooling the platelets to a temperature of about −80° C.;
   (f) storing the platelets at about −80° C. for a period of time; and
   (g) thawing the platelets at about 20-40° C.
Clause 71. Activated recovered cryopreserved platelets prepared by the following method:
   (a) obtaining blood, platelet-rich plasma, or apheresis-derived platelets from a subject;
   (b) isolating platelets from the blood, platelet-rich plasma, or apheresis platelet;
   (c) suspending the platelets in a buffer solution comprising trehalose;
   (d) adding a stabilizing agent to the suspended platelets;
   (e) cooling the platelets to a temperature of about −80° C.;
   (f) storing the platelets at about −80° C. for a period of time;
   (g) thawing the platelets at about 20-40° C.;
   (h) isolating the thawed platelets;
   (i) suspending the isolated thawed platelets in an activation buffer comprising BSA at about 1% by mass;
   (j) treating the recovered platelets with heparin or PF4; and
   (k) incubating the heparin- or PF4-treated platelets with a plasma, serum, or blood sample from a subject diagnosed with heparin induced thrombocytopenia (HIT).
Clause 72. Activated recovered cryopreserved platelets prepared by the following method: Clause 72.
   (a) obtaining blood, platelet-rich plasma, or apheresis-derived platelets from a subject;
   (b) isolating platelets from the blood, platelet-rich plasma, or apheresis platelet;
   (c) suspending the platelets in a buffer solution comprising trehalose;
   (d) adding a stabilizing agent to the suspended platelets;
   (e) cooling the platelets to a temperature of about −80° C.;
   (f) storing the platelets at about −80° C. for a period of time;
   (g) thawing the platelets at about 20-40° C.;
   (h) isolating the thawed platelets;
   (i) suspending the isolated thawed platelets in an activation buffer comprising BSA at about 1% by mass; and
   (j) treating the recovered platelets with Thrombin Receptor Activating Peptide (TRAP; amino acid sequence: SFFLRN; SEQ ID NO: 1).
Clause 73. A kit for the detection of heparin-induced thrombocytopenia (HIT) antibodies in blood, plasma or serum, the kit comprising two or more of:
   (a) cryopreserved platelets;
   (b) one or more of platelet factor 4 (PF4; SEQ ID NO: 2) or heparin;
   (c) one or more reagents for detecting platelet activation;
   (d) optionally, buffers and receptacles; and
   (e) optionally, one or more of packaging or instruction for use.
Clause 74. The kit of clause 73, wherein the one or more reagents for detecting platelet activation comprise reagents for the detection of thrombospondin-1.
Clause 75. The kit of clause 73 or 74, wherein the kit further comprises one or more negative control samples negative for platelet-activating pathogenic HIT antibodies; and one or more positive control samples positive for platelet-activating pathogenic HIT antibodies.
Clause 76. A method for treating or prophylaxis of hemorrhage in a subject in need thereof, the method comprising:
   (a) obtaining cryopreserved platelets;
   (b) thawing the cryopreserved platelets;
   (c) contacting the subject with the thawed cryopreserved platelets.
Clause 77. The method of clause 76, wherein steps (a)-(b) comprise:
   (i) obtaining blood, platelet-rich plasma, or apheresis-derived platelets from a second subject;
   (ii) isolating platelets from the blood, platelet-rich plasma, or apheresis platelets;
   (iii) suspending the platelets in a buffer solution comprising trehalose;
   (iv) incubating trehalose-treated platelets at 37° C. for about 2 hours;
   (v) adding a stabilizing agent to the suspended platelets;
   (vi) cooling the platelets to a temperature of about −80° C.;
   (vii) storing the platelets at about −80° C. for a period of time; and
   (viii) thawing the platelets at about 20-40° C.
Clause 78. The method of clause 76, wherein steps (a)-(b) comprise:
   (i) obtaining platelet-rich plasma or apheresis-derived platelets from a second subject;
   (ii) adding trehalose to the platelet rich plasma or apheresis-derived platelets;
   (iii) incubating trehalose-treated platelets at 37° C. for about 2 hours;
   (iv) cooling the platelets to a temperature of about −80° C.;
   (v) storing the platelets at about −80° C. for a period of time; and
   (vi) thawing the platelets at about 20-40° C.
Clause 79. The method of any one of clauses 76-78, wherein the contacting in step (c) comprises applying the thawed cryopreserved platelets to an injury situs or parenterally administering the thawed cryopreserved platelets to the subject.
Clause 80. Use of cryopreserved platelets as a reagent, research tool, or as a medicament for treating a subject in need thereof.
Clause 81. Use of thawed cryopreserved platelets as a reagent, research tool, or as a medicament for treating a subject in need thereof.
Clause 82. Use of activated, thawed cryopreserved platelets as a reagent, research tool, or as a medicament for treating a subject in need thereof.

### EXAMPLES

### Example 1

### Cryopreservation of Platelets in Buffer

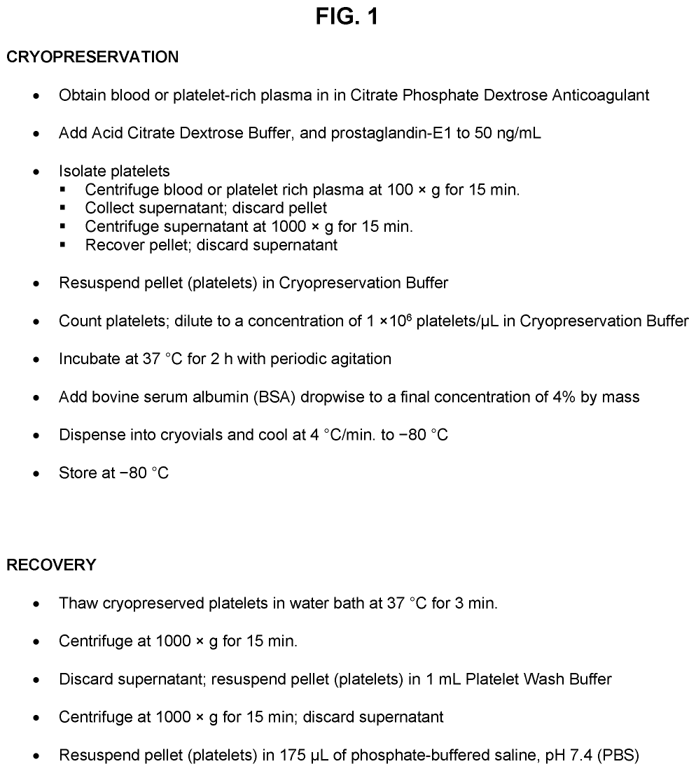

Platelet-rich plasma was collected (45 mL) in Citrate Phosphate Dextrose Anticoagulant (CPD, 15.6 mM citric acid, 89 mM sodium citrate dihydrate, 16 mM NaH₂PO₄·H₂O, 128 mM dextrose monohydrate) and 5 mL of Acid Citrate Dextrose Buffer (117 mM sodium citrate, 136 mM dextrose, pH 5.9; 1:10 final dilution) was added with prostaglandin-E1 (final concentration 50 ng/ml). The combination was centrifuged at 100×g for 15 minutes. The supernatant (40 mL) was collected and centrifuged at 1000×g for 15 minutes and the supernatant was removed and discarded. The platelets were resuspended in Cryopreservation Buffer (9.5 mM HEPES; 100 mM NaCl; 4.8 mM KCl; 5.0 mM glucose; 12 mM NaHCO₃; 50 mM trehalose, pH 6.8). The platelets were counted and diluted to a final concentration of 1×10⁶ platelets/μL with Cryopreservation Buffer. The platelets were incubated at 37° C. for 2 hours, with inversion every 30 minutes. After 2-hours incubation, bovine serum albumin (BSA) was added dropwise to a final concentration of 4% by mass. Aliquots of 1 mL were dispensed into cryovials and cooled at a rate of 4° C./minute to a final temperature of −80° C. The vials were transferred to a −80° C. freezer for long-term storage. See FIG. 1.

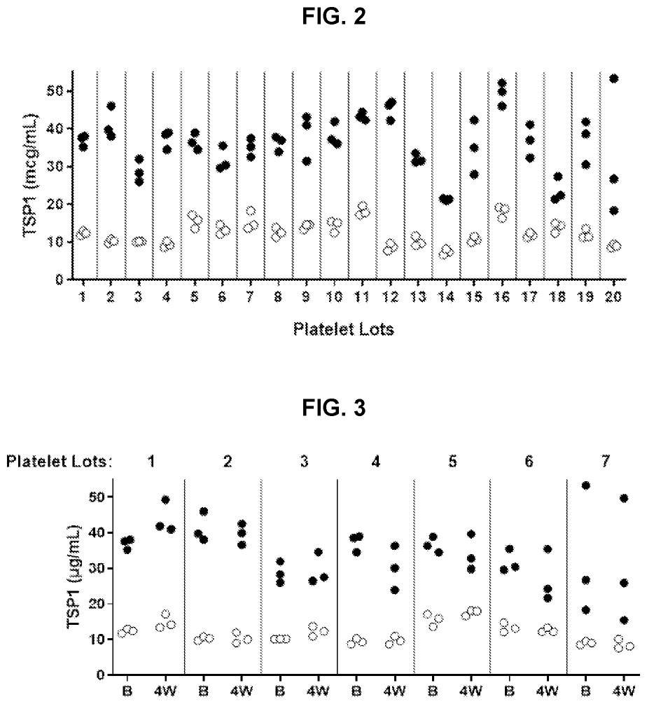

Platelets from 20 different whole blood donors were obtained and cryopreserved in buffer, as described. Cryopreserved platelets were stored at −80° C. until use and recovered by washing. After recovery, platelets were treated with platelet factor 4 (PF4) and were added to one of three blood samples from patients with confirmed heparin induced thrombocytopenia (HIT), or one of three normal control sera. After treatment of the PF4 treated platelets with patient/normal sample, supernatant was collected and assayed for thrombospondin-1 concentration using a thrombospondin-1 (TSP1) ELISA kit. As shown in FIG. 2, each of the 20 different platelet lots tested clearly distinguished the three HIT serum sample from the three normal serum samples tested.

Of the 20 different cryopreserved platelet lots shown in FIG. 2, seven were tested after four or more weeks of storage at −80° C. Cryopreserved platelets were recovered by washing, PF4 treated and activated by HIT or normal blood samples. As shown in FIG. 3, all platelets lots tested after four weeks of storage clearly distinguished HIT from normal samples tested.

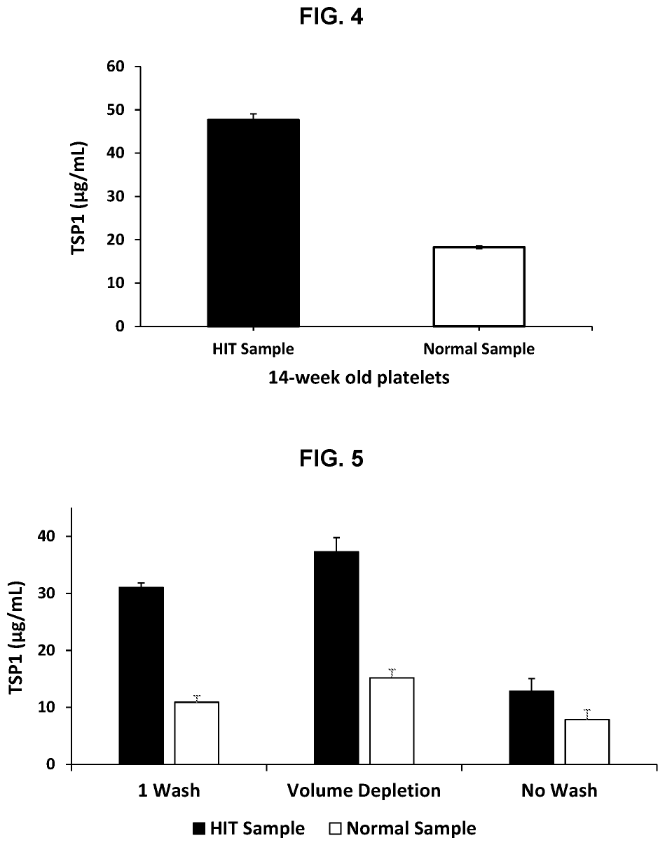

Platelets from one of the cryopreserved lots shown in FIG. 2 were tested after 14 weeks of storage at −80° C. Cryopreserved platelets were recovered by washing, PF4 treated and activated a HIT or normal serum sample. As shown in FIG. 4, after 14-weeks of storage, the cryopreserved platelets retain their ability to be activated.

### Example 2

### Recovery of Cryopreserved Platelets by Washing

Cryopreserved 1 mL aliquots of platelets were thawed in a 37° C. water bath for 3 minutes. The platelets were then centrifuged at 1000×g for 15 minutes and the supernatant was removed and discarded. The platelets were resuspended in 1 mL Platelet Wash Buffer (108 mM NaCl, 3.8 mM KCl, 1.7 mM NaHCO₃, 22.9 mM sodium citrate, 27.8 mM glucose, pH 6.5). The platelets were centrifuged at 1000×g for 15 minutes and the supernatant was removed and discarded. The platelets were resuspended in 175 μL phosphate buffered saline (PBS, 137 mM NaCl, 2.7 mM KCl, 4.3 mM Na₂HPO₄, 1.47 mM KH₂PO₄, PH 7.4) containing 1% BSA. See FIG. 1.

### Platelet Factor 4 (PF4) Treatment and Activation of Recovered-Cryopreserved Platelets with Blood Samples from Patients with Heparin Induced Thrombocytopenia (HIT)

PF4 (SEQ ID NO: 2) was added to recovered, washed, cryopreserved platelets at a concentration of 150 μg/mL and incubated 20 minutes at room temperature. An 80 μL aliquot of PF4-treated platelets was added to 20 μL of HIT patient serum samples or normal serum samples and incubated 30 minutes at room temperature. The platelet reaction mixture was centrifuged at 1000×g for 15 minutes and 40 μL of supernatant was collected for assessment of thrombospondin-1 (a platelet alpha granule component) concentration as a marker of platelet activation.

### Measurement of Thrombospondin-1

A commercially-available thrombospondin-1 (TSP1) ELISA kit (R&D Systems) was used to quantify TSP1 in platelet supernatants as a measure of platelet activation. Briefly, the ELISA plate was coated with 1 μg/mL anti-TSP1 capture antibody at 4° C. for at least 8 hours. ELISA plate was washed 3 times with 400 μL ELISA plate wash buffer and blocked with ELISA reagent diluent. Supernatants from platelet reactions were diluted 1:1000 or 1:2000. Standards were prepared according to manufacturer instructions and incubated on the ELISA plate. The ELISA plate was washed 3 times with 400 μL ELISA plate wash buffer and incubated with 100 ng/ml biotin labeled detection antibody. The ELISA plate was washed again 3 times with 400 μL ELISA plate wash buffer and incubated with 1:200 horseradish peroxidase labeled streptavidin. The ELISA plate was washed again 3 times with 400 μL ELISA plate wash buffer and incubated with tetramethylbenzidine. The reaction was terminated after 10 minutes with H₂SO₄ and the optical density at 450 nm was measured. TSP1 concentrations were determined using a four-parameter logistic regression against a standard curve.

### Recovery of Cryopreserved Platelets by Volume Depletion

Cryopreserved 1 mL aliquots of platelets were thawed in 37° C. water bath for three minutes. The platelets were centrifuged at 1000×g for 15 minutes and the supernatant removed and discarded. The platelets were resuspended in 175 μL PBS, pH 7.4 containing 1% BSA by mass.

### Recovery of Cryopreserved Platelets by Thawing

Cryopreserved 1 mL aliquots of platelets were thawed in 37° C. water bath for three minutes. The platelets were used without further treatment.

### Activation of Recovered-Cryopreserved Platelets with Thrombin Receptor Activating Peptide (TRAP)

An 80 μL aliquot of recovered-cryopreserved platelets was added to 20 μL of 125 g/ml TRAP peptide (TRAP final concentration: 25 μg/mL; amino acid sequence: SFFLRN; SEQ ID NO: 1) or 20 μL pH 7.4 PBS containing 1% BSA (control) and incubated for 30 minutes at room temperature. The platelets reaction mixture was centrifuged at 1000×g for 15 minutes and 40 μL of the supernatant was collected for assessment of thrombospondin-1 concentration as a marker of platelet activation.

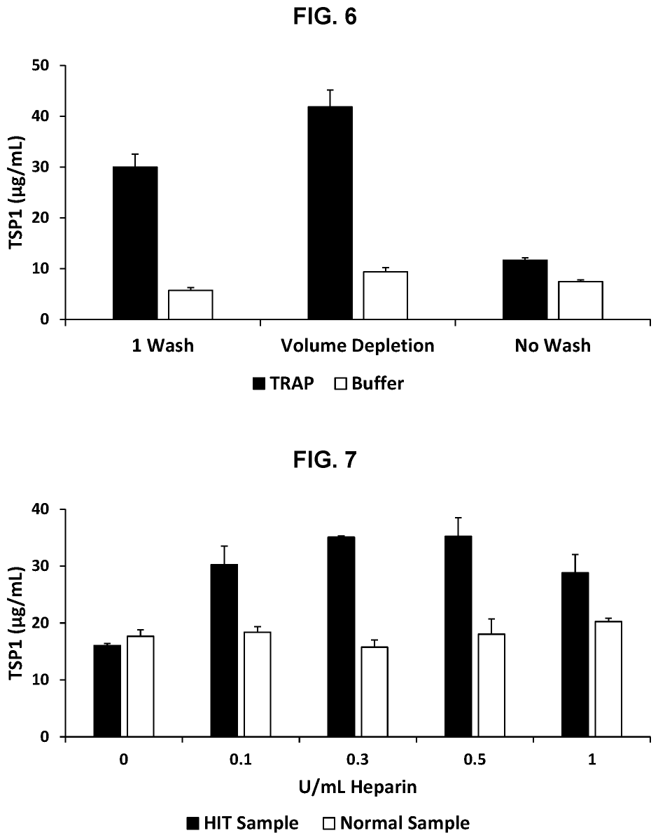

The method of recovery of cryopreserved platelets was also investigated. Platelets from a single donor were cryopreserved in buffer. Cryopreserved platelets were either washed, volume depleted, or used directly upon thawing. After recovery, platelets were PF4 treated and activated by a HIT or normal serum sample. Additionally, recovered platelets were treated with thrombin receptor activating peptide (TRAP), a strong platelet agonist, or buffer as a control. Both human samples (FIG. 5), and the TRAP (FIG. 6) cryopreserved platelets that were washed, or volume depleted have a higher degree of activatability than cryopreserved platelets that are solely thawed.

### Example 3

### Heparin Treatment and Activation of Recovered-Cryopreserved Platelets with Blood Samples from Patients with Heparin Induced Thrombocytopenia (HIT)

Unfractionated heparin was added to recovered washed, cryopreserved platelets at concentrations of 0.1, 0.3, 0.5, 1 Units/mL or 0 U/mL as a control and the mixtures were incubated for 20 minutes at room temperature. An 80 μL aliquot of the heparin treated recovered-cryopreserved platelets was added to 20 μL of HIT patient serum samples or normal patient serum samples and incubated for 30 minutes at room temperature. The platelets' reaction mixture was centrifuged at 1000×g for 15 minutes and 40 μL of the supernatant was collected for assessment of thrombospondin-1 concentration as a marker of platelet activation.

FIG. 7 demonstrates that heparin-treated, cryopreserved platelets can be used to differentiate HIT from normal blood samples, whereby, the HIT sample activates platelets while the normal sample does not.

### Example 4

### Cryopreservation of Platelets in the Presence of Platelet Poor Plasma

Platelet rich plasma (45 mL) was collected in Citrate Phosphate Dextrose Anticoagulant (CPD) and 5 mL of Acid Citrate Dextrose Buffer was added with prostaglandin-E1 (final concentration 50 ng/ml). The platelet rich plasma mixture was centrifuged at 100×g for 15 minutes. The supernatant (40 mL) was collected and centrifuged 1000×g for 15 minutes. The supernatant (platelet poor plasma, PPP) was removed and used as described below. The platelets were resuspended in 1 mL Cryopreservation Buffer (9.5 mM HEPES; 100 mM NaCl; 4.8 mM KCl; 5.0 mM glucose; 12 mM NaHCO₃; 50 mM trehalose, pH 6.8). Trehalose (200 μL of 1 M trehalose) was added to 4 mL of PPP to make PPP containing 50 mM Trehalose. The platelets were counted and diluted to a final concentration of 1×10⁶ platelets/μL with PPP containing 50 mM trehalose. The platelets were incubated at 37° C. for 2 hours, with inversion every 30 minutes. After a 2-hour incubation, 1 mL aliquots of platelets were dispensed into cryovials and cooled at a rate of 4° C./min to a final temperature of −80° C. The vials were transferred to a −80° C. freezer for long-term storage.

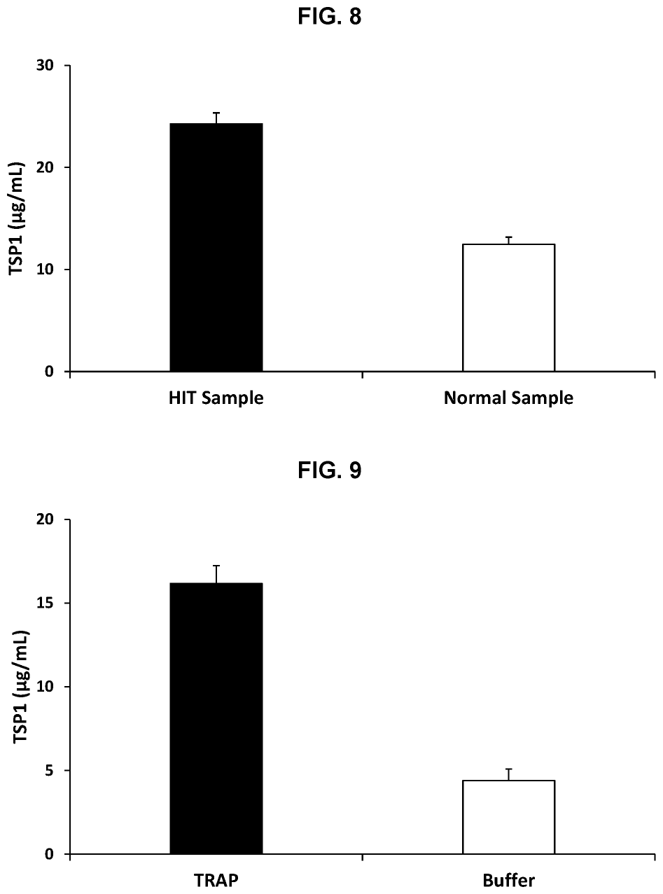

Platelets cryopreserved in PPP were then recovered by washing. After recovery, platelets were PF4-treated and activated by HIT or normal serum. Additionally, recovered platelets were treated with TRAP or buffer as a control. Similar to platelets cryopreserved in buffer, platelets cryopreserved in PPP are capable of being activated by HIT antibodies (FIG. 8) or TRAP (FIG. 9).

### Example 5

### Lyophilization of Cryopreserved Platelets

Platelets were cryopreserved in buffer, as discussed above in Example 1, and the vials were transferred to a lyophilizer equilibrated to −40° C. at atmospheric pressure for 4 hours. Vacuum was applied (100 mTorr) at −30° C. for 13 hours. Afterwards, with 100 mTorr vacuum applied, the temperature was gradually increased from −30° C. to +30° C. over a period of 12 hours. The vials were then transferred to a −80° C. freezer.

### Reconstitution, PF4 Treatment and Treatment of Lyophilized Platelets with HIT and Normal Blood Samples

A 1 mL aliquot of H₂O was added to each lyophilized platelet aliquot. PF4 was added at a concentration of 150 μg/mL to the reconstituted lyophilized platelets and incubated for 20 minutes at room temperature. An 80 μL aliquot of PF4 treated-cryopreserved platelets was added to 20 μL of HIT or normal serum and incubated for 30 minutes at room temperature. The platelet reaction mixture was centrifuged at 1000×g for 15 minutes and 40 μL of supernatant was collected for assessment of thrombospondin-1 concentration as a marker of platelet activation.

### Reconstitution and Activation of Lyophilized Platelets with TRAP

Lyophilized platelets were reconstituted with 1 mL of H₂O. An 80 μL aliquot of the reconstituted platelets was added to 20 μL of 125 μg/mL TRAP peptide (TRAP final concentration: 25 μg/mL; amino acid sequence: SFFLRN; SEQ ID NO: 1) or 20 UL PH 7.4 PBS containing 1% BSA by mass (control) and incubated for 30 minutes at room temperature. The platelet reaction mixture was centrifuged at 1000×g for 15 minutes and 40 μL of the supernatant was collected for assessment of thrombospondin-1 concentration as a marker of platelet activation.

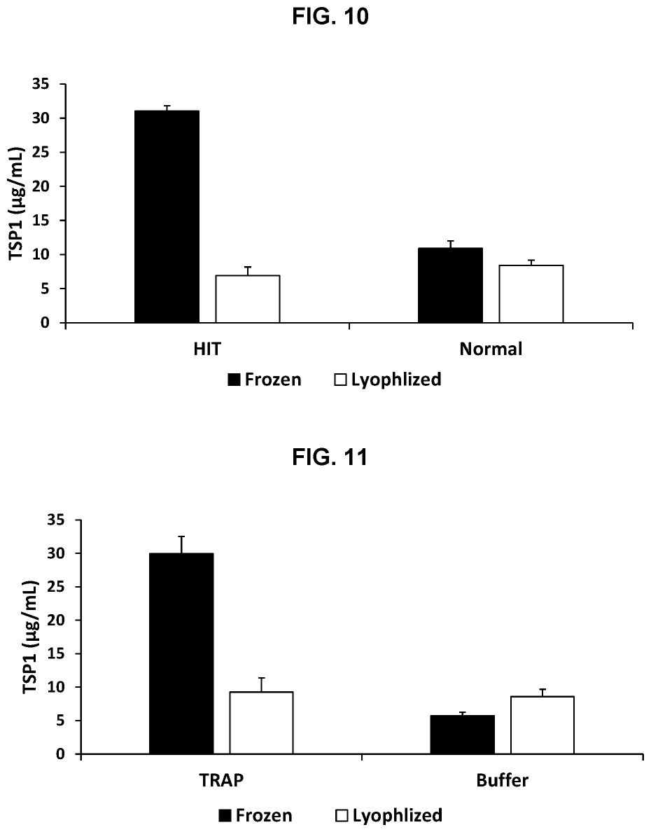

Lyophilization is another commonly used technique to cryopreserve cells. Platelets from a single donor were cryopreserved in buffer and a subset of aliquots were subjected to lyophilization. Frozen platelets were recovered by washing, and lyophilized platelets were reconstituted with H₂O. After recovery, lyophilized or frozen platelets were PF4 treated and activated by a HIT or normal serum sample. Additionally, platelets were treated with TRAP or buffer as a control. Unlike frozen platelets, lyophilized platelets have no ability to be activated by HIT samples (FIG. 10) or by TRAP (FIG. 11).

### Example 6

### Modified Recovery, Platelet Factor 4 (PF4) Treatment and Activation of Cryopreserved Platelets with Blood Samples from Patients with Heparin Induced Thrombocytopenia (HIT)

Modified recovery refers to (1) different centrifugation conditions (5 min. vs 15 min. as discussed above), and (2) the use of trehalose in resuspension buffers (wash buffer and activation buffer) relative to the use of trehalose only in the cryopreservation buffer used for freezing the platelets.

Platelets were cryopreserved in a 500 μL volume at a concentration of 1×10⁶ platelets/μL and thawed in a 37° C. water bath for three minutes. The platelets were centrifuged at 1000×g for 5 minutes and the supernatant was removed and discarded. The platelets were resuspended with 500 μL Platelet Wash Buffer containing 50 mM trehalose. The platelets were centrifuged at 1000×g for 5 minutes and the supernatant was removed and discarded. The platelets were resuspended with 80 μL PBS pH 7.4 containing 1% BSA by mass, 50 mM trehalose, and PF4 (SEQ ID NO: 2) at 150 μg/mL, and the mixture incubated for 20 minutes at room temperature. A 20 μL aliquot of HIT patient serum sample or normal patient serum sample was added, and the mixture incubated for an additional 30 minutes at room temperature. The platelet reaction mixture was centrifuged at 1000×g for 5 minutes and 40 μL of the supernatant was collected for assessment of thrombospondin-1 (a platelet alpha granule component) concentration as a marker of platelet activation.

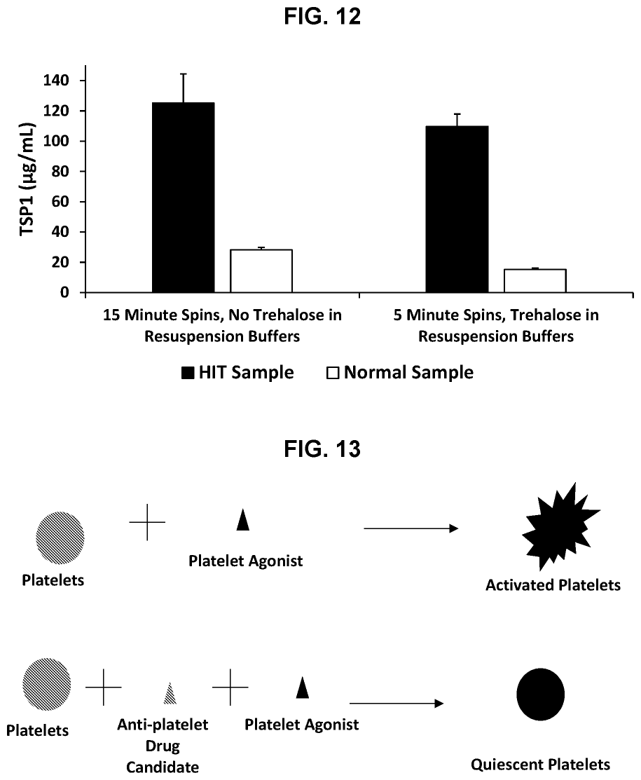

The modified activation procedure was also employed to enhance the signal (HIT) to background (Normal) ratio obtained with frozen platelets. In brief, platelets were cryopreserved in the presence of 50 mM trehalose, then recovered by washing with RCD containing 50 mM trehalose and resuspended in PBS containing 1% BSA by mass, 50 mM trehalose, and PF4 (SEQ ID NO: 2) at 150 g/mL. Additionally, the centrifugation time for all centrifugation steps post-thaw was changed from 15 minutes to 5 minutes. FIG. 12 compares the standard activation procedure (left) to this modified procedure (right). The signal to background ratio is 4.4-fold with the standard procedure and 7.2-fold with the modified procedure.

In addition to demonstrating the utility of frozen platelets in detection of samples with platelet-activating HIT antibodies, FIG. 13 presents a model by which these platelets can be used for anti-platelet drug discovery.

### Example 7

### Therapeutic Use of Cryopreserved Platelets

Platelet transfusion are lifesaving in the setting of prophylaxis and treatment of bleeding in a variety of conditions including but not limited to trauma, cancer, and autoimmune disorders. Donor platelets intended for transfusion are typically stored at room temperature and have a shelf-life of 5-7 days. They can be stored for slightly longer periods (2 weeks) at 4° C. This limitation to platelet shelf-life based on current technologies creates logistical challenges in treating bleeding patients or thrombocytopenia patients at risk for bleeding, particularly in resource-poor environments such as rural areas, conflict zones, developing countries and during periods of blood shortages as seen frequently after natural disasters and inclement weather. Lyophilized platelets, which have a long shelf life (months to years), have been proposed as an alternative, however, while these platelet preparations are already in an activated state and expose surface phospholipids on which thromboses can form, they are suboptimal as they do not degranulate upon stimulation. Platelet granules contain key molecules that can further activate platelets, such as ADP, as well as other components that regulate the coagulation cascade. Thus, there is a critical need to develop a platelet product that not only has long shelf-life, but one that, like fresh platelets, is capable of degranulation upon activation.

Platelets with a long shelf-life capable of degranulation that are intended for transfusion will be prepared similarly to the diagnostic platelet preparation described in this application, with some changes as noted below.

### Platelet Source and Platelet Isolation

There are a number of methods for the collection and isolation of platelets for cryopreservation and therapeutic use. Platelets are collected via apheresis methods, or platelet rich plasma components are produced by centrifugation techniques from standard whole blood collection.

### Preparing Platelets for Cryopreservation

Trehalose is added to the platelet product to achieve a concentration of approximately 50 mM. This can be achieved in several ways. For example, platelets can be collected directly into a blood bag containing trehalose; this can be done with apheresis platelets, or with platelet rich plasma isolated by centrifugation from a whole blood unit. Another method of introducing trehalose into platelets is by adding a concentrated trehalose solution to isolated platelets obtained by apheresis or by production of platelet rich plasma from whole blood. Alternatively, collected platelets can be pelleted by centrifugation of apheresis platelets or platelet rich plasma platelets and the pellet resuspended in a trehalose containing buffer, similar to the procedure described herein for the diagnostic cryopreserved platelet product. After the addition of trehalose, platelets are incubated for about 2 hours at about 37° C.

If the trehalose-treated platelets are in platelet rich plasma or in apheresis-derived plasma (which already contain human albumin), no further addition of human albumin is needed. If platelets are pelleted and resuspended in platelet poor plasma (which already contain human albumin), no further addition of human albumin is needed. Human albumin at about a 4-5% final concentration is added to trehalose-treated platelet preparation if the platelet pellet is resuspended in a trehalose containing albumin-free buffer.

### Cryopreservation

Platelets prepared by any of the methods described herein are cooled to about −80° C. either gradually at a rate of 2-20° C./min or in an uncontrolled fashion. Prior to freezing, it may be desirable, but not necessary, to concentrate platelets by centrifugation and discard some of the platelet-free supernatant in order to decrease the volume of platelets to be frozen, and thus the volume of platelets that would be transfused into a patient. Platelets can be frozen in bags or in tubes.

### Thawing and Therapeutic Use

Cryopreserved platelets are thawed at 20-40° C. Depending on the volume of platelets frozen, this could take between 5 minutes and an hour. Thawed platelets may be directly infused parenterally into patients prophylactically, such as in a thrombocytopenic patient at elevated risk of bleeding, or therapeutically in patients who are actively hemorrhaging.

What is claimed:

1. A method for cryopreserving platelets that maintains viability and activation potential, the method comprising:
(a) obtaining blood or platelet-rich plasma, or apheresis-derived platelets from a subject;
(b) isolating platelets from the blood, platelet-rich plasma, or apheresis platelets;
(c) suspending the platelets in a suspension buffer comprising trehalose;
(d) adding a stabilizing agent to the suspended platelets;
(e) cooling the platelets to a temperature of about −80° C.; and
(f) storing the platelets at about −80° C.

2. The method of claim 1, wherein the method maintains the viability and activation potential of the platelets for greater than or equal to 2 weeks.

3. The method of claim 1, wherein the suspension buffer comprises about 50 mM trehalose.

4. The method of claim 1, wherein the stabilizing agent is bovine serum albumin (BSA) at about 4% by mass.

5. The method of claim 1, wherein the platelets of step (c) are diluted with suspension buffer to a final concentration of about 1×10⁶ platelets/μL.

6. The method of claim 5, wherein the diluted platelets are incubated at about 37° C. for about 2 hours with periodic agitation.

7. The method of claim 1, wherein isolating the platelets comprises centrifugation of the blood, platelet-rich plasma, or apheresis platelets at 100×g for 15 minutes to generate a first pellet and a first supernatant; followed by centrifugation of the first supernatant at 1000×g for 15 minutes to generate a second pellet and a second supernatant, wherein the first supernatant is platelet-rich and the second supernatant is platelet-poor.

8. The method of claim 7, wherein the second supernatant is collected, trehalose is added to about 50 mM, and is used to dilute the platelets of step (c) to a concentration of about 1×10⁶ platelets/μL.

9. The method of claim 1, wherein cooling the platelets comprises decreasing the temperature at a rate of about 2-20° C. per minute.

10. Cryopreserved platelets prepared by the method of claim 1.

11. A method for cryopreserving and recovering platelets from cryopreservation, the method comprising:
(a) obtaining blood, platelet-rich plasma, or apheresis-derived platelets from a subject;
(b) isolating platelets from the blood, platelet-rich plasma, or apheresis platelets;
(c) suspending the platelets in a buffer solution comprising trehalose;
(d) adding a stabilizing agent to the suspended platelets;
(e) cooling the platelets to a temperature of about −80° C.;
(f) storing the platelets at about −80° C. for a period of time; and
(g) thawing the platelets at about 20-40° C.

12. The method of claim 11, wherein the suspension buffer comprises about 50 mM trehalose.

13. The method of claim 11, wherein the stabilizing agent is bovine serum albumin (BSA) at about 4% by mass.

14. The method of claim 11, wherein the platelets of step (c) are diluted with suspension buffer to a final concentration of about 1×10⁶ platelets/μL.

15. The method of claim 14, wherein the diluted platelets are incubated at about 37° C. for about 2 hours with periodic agitation.

16. The method of claim 11, further comprising the steps:
(h) isolating the thawed platelets; and
(i) suspending the isolated thawed platelets in an activation buffer comprising BSA at about 1% by mass.

17. The method of claim 16, wherein isolating the thawed platelets comprises centrifugation at 1000×g for 15 minutes.

18. The method of claim 16, wherein the activation buffer further comprises about 50 mM trehalose.

19. The method of claim 18, wherein isolating the thawed platelets comprises centrifugation at 1000×g for 5 minutes.

20. The method of claim 16, wherein after step (h) and prior to step (i), the following step is performed:
(h-1) washing the thawed platelets in a wash buffer.

21. The method of claim 20, wherein isolating the thawed platelets in step (h) comprises centrifugation at 1000×g for 15 minutes and removal of the supernatant; and washing the thawed platelets in step (h-1) comprises resuspending platelets in wash buffer, centrifugation at 1000×g for 15 minutes and removal of supernatant.

22. The method of claim 20, wherein the wash buffer further comprises about 50 mM trehalose.

23. The method of claim 20, wherein isolating the thawed platelets in step (h) comprises centrifugation at 1000×g for 5 minutes and removal of the supernatant; and washing the thawed platelets in step (h-1) comprises resuspending platelets in wash buffer, centrifugation at 1000×g for 5 minutes and removal of supernatant.

24. The method of claim 16, wherein the period of time in step (f) is from 1 day to about 5 years.

25. Recovered cryopreserved platelets prepared by the method of claim 11.

26. A method for activation of recovered platelets after cryopreservation, the method comprising:
(a) recovering platelets after cryopreservation according to the method of claim 11;
(b) treating the recovered platelets with platelet factor 4 (PF4; SEQ ID NO: 2); and
(c) incubating the PF4-treated platelets with a plasma, serum, or blood sample from a subject diagnosed with heparin induced thrombocytopenia (HIT).

27. The method of claim 26, wherein treating the recovered platelets with PF4 comprises adding PF4 to the platelets at a concentration of about 150 μg/mL and incubating for about 20 minutes at room temperature.

28. The method of claim 26, wherein activation of the platelets is assessed by measuring platelet granule content released after incubation of the PF4-treated platelets with the plasma, serum, or blood sample from the HIT subject.

29. The method of claim 28, wherein the platelet granule content is thrombospondin-1.

30. Activated recovered cryopreserved platelets prepared by the method of claim 26.

31. A method for activation of recovered platelets after cryopreservation, the method comprising:
(a) recovering platelets after cryopreservation according to the method of claim 11; and
(b) treating the recovered platelets with Thrombin Receptor Activating Peptide (TRAP; amino acid sequence: SFFLRN; SEQ ID NO: 1).

32. The method of claim 31, wherein treating the recovered platelets with TRAP comprises adding TRAP to the platelets at a concentration of about 25 μg/mL and incubating for about 30 minutes at room temperature.

33. The method of claim 31, wherein activation of the platelets is assessed by measuring platelet granule content released after treating the recovered platelets with TRAP.

34. The method of claim 33, wherein the platelet granule content is thrombospondin-1.

35. Activated recovered cryopreserved platelets prepared by the method of claim 31.

36. A method for activation of recovered platelets after cryopreservation, the method comprising:
(a) recovering platelets after cryopreservation according to the method of claim 11;
(b) treating the recovered platelets with heparin; and
(c) incubating the heparin-treated platelets with a plasma, serum, or blood sample from a subject diagnosed with heparin induced thrombocytopenia (HIT).

37. The method of claim 36, wherein treating the recovered platelets with heparin comprises adding heparin to the platelets at a concentration of about 0.1-5 U/mL and incubating for about 20 minutes at room temperature.

38. The method of claim 36, wherein activation of the platelets is assessed by measuring platelet granule content released after incubation of the heparin-treated platelets with the plasma, serum, or blood sample from the HIT subject.

39. The method of claim 38, wherein the platelet granule content is thrombospondin-1.

40. Activated recovered cryopreserved platelets prepared by the method of claim 36.

41. A method for cryopreserving platelets and recovering platelets from cryopreservation, the method comprising:
(a) obtaining blood, platelet-rich plasma, or apheresis-derived platelets from a subject;
(b) isolating platelets from the blood, platelet-rich plasma, or apheresis platelet;
(c) suspending the platelets in a buffer solution comprising trehalose;
(d) adding a stabilizing agent to the suspended platelets;
(e) cooling the platelets to a temperature of about −80° C.;
(f) storing the platelets at about −80° C. for a period of time;
(g) thawing the platelets at about 20-40° C.;
(h) isolating the thawed platelets; and
(i) suspending the isolated thawed platelets in an activation buffer comprising BSA at about 1% by mass.

42. The method of claim 41, wherein the suspension buffer comprises about 50 mM trehalose.

43. The method of claim 41, wherein the stabilizing agent is bovine serum albumin (BSA) at about 4% by mass.

44. The method of claim 41, wherein the platelets of step (c) are diluted with suspension buffer to a final concentration of about 1×10⁶ platelets/μL.

45. The method of claim 44, wherein the diluted platelets are incubated at about 37° C. for about 2 hours with periodic agitation.

46. The method of claim 41, wherein isolating the thawed platelets comprises centrifugation at 1000×g for 15 minutes.

47. The method of claim 41, wherein the activation buffer further comprises about 50 mM trehalose.

48. The method of claim 41, wherein isolating the thawed platelets comprises centrifugation at 1000×g for 5 minutes.

49. The method of claim 41, wherein after step (h) and prior to step (i), the following step is performed:
(h-1) washing the thawed platelets in a wash buffer.

50. The method of claim 49, wherein isolating the thawed platelets in step (h) comprises centrifugation at 1000×g for 15 minutes and removal of the supernatant; and washing the thawed platelets in step (h-1) comprises resuspending platelets in wash buffer, centrifugation at 1000×g for 15 minutes and removal of supernatant.

51. The method of claim 49, wherein the wash buffer further comprises about 50 mM trehalose.

52. The method of claim 51, wherein isolating the thawed platelets in step (h) comprises centrifugation at 1000×g for 5 minutes and removal of the supernatant; and washing the thawed platelets in step (h-1) comprises resuspending platelets in wash buffer, centrifugation at 1000×g for 5 minutes and removal of supernatant.

53. The method of claim 41, wherein the period of time in step (f) is from 1 day to about 5 years.

54. Recovered cryopreserved platelets prepared by the method of claim 41.

55. A method for activation of recovered platelets after cryopreservation, the method comprising:
(a) recovering platelets after cryopreservation according to the method of claim 41;
(b) treating the recovered platelets with platelet factor 4 (PF4; SEQ ID NO: 2); and
(c) incubating the PF4-treated platelets with a plasma, serum, or blood sample from a subject diagnosed with heparin induced thrombocytopenia (HIT).

56. The method of claim 55, wherein treating the recovered platelets with PF4 comprises adding PF4 to the platelets at a concentration of about 150 μg/mL and incubating for about 20 minutes at room temperature.

57. The method of claim 55, wherein activation of the platelets is assessed by measuring platelet granule content released after incubation of the PF4-treated platelets with the plasma, serum, or blood sample from the HIT subject.

58. The method of claim 57, wherein the platelet granule content is thrombospondin-1.

59. Activated recovered cryopreserved platelets prepared by the method of claim 55.

60. A method for activation of recovered platelets after cryopreservation, the method comprising:
(a) recovering platelets after cryopreservation according to the method of claim 41; and
(b) treating the recovered platelets with Thrombin Receptor Activating Peptide (TRAP; amino acid sequence: SFFLRN; SEQ ID NO: 1).

61. The method of claim 60, wherein treating the recovered platelets with TRAP comprises adding TRAP to the platelets a concentration of about 25 μg/mL and incubating for about 30 minutes at room temperature.

62. The method of claim 60, wherein activation of the platelets is assessed by measuring platelet granule content released after treating the recovered platelets with TRAP.

63. The method of claim 62, wherein the platelet granule content is thrombospondin-1.

64. Activated recovered cryopreserved platelets prepared by the method of claim 60.

65. A method for activation of recovered platelets after cryopreservation, the method comprising:
(a) recovering platelets after cryopreservation according to the method of claim 41;
(b) treating the recovered platelets with heparin; and
(c) incubating the heparin-treated platelets with a plasma, serum, or blood sample from a subject diagnosed with heparin induced thrombocytopenia (HIT).

66. The method of claim 65, wherein treating the recovered platelets with heparin comprises adding heparin to the platelets at a concentration of about 0.1-5 U/mL and incubating for about 20 minutes at room temperature.

67. The method of claim 65, wherein activation of the platelets is assessed by measuring platelet granule content released after incubation of the heparin-treated platelets with the plasma, serum, or blood sample from the HIT subject.

68. The method of claim 67, wherein the platelet granule content is thrombospondin-1.

69. Activated recovered cryopreserved platelets prepared by the method of claim 65.

70. Recovered cryopreserved platelets produced by the following method:
(a) obtaining blood, platelet-rich plasma, or apheresis-derived platelets from a subject;
(b) isolating platelets from the blood, platelet-rich plasma, or apheresis platelets;
(c) suspending the platelets in a buffer solution comprising trehalose;
(d) adding a stabilizing agent to the suspended platelets;
(e) cooling the platelets to a temperature of about −80° C.;
(f) storing the platelets at about −80° C. for a period of time; and
(g) thawing the platelets at about 20-40° C.

71. Activated recovered cryopreserved platelets prepared by the following method:
(a) obtaining blood, platelet-rich plasma, or apheresis-derived platelets from a subject;
(b) isolating platelets from the blood, platelet-rich plasma, or apheresis platelet;
(c) suspending the platelets in a buffer solution comprising trehalose;
(d) adding a stabilizing agent to the suspended platelets;
(e) cooling the platelets to a temperature of about −80° C.;
(f) storing the platelets at about −80° C. for a period of time;
(g) thawing the platelets at about 20-40° C.;
(h) isolating the thawed platelets;
(i) suspending the isolated thawed platelets in an activation buffer comprising BSA at about 1% by mass;
(j) treating the recovered platelets with heparin or PF4; and
(k) incubating the heparin- or PF4-treated platelets with a plasma, serum, or blood sample from a subject diagnosed with heparin induced thrombocytopenia (HIT).

72. Activated recovered cryopreserved platelets prepared by the following method:
(a) obtaining blood, platelet-rich plasma, or apheresis-derived platelets from a subject;
(b) isolating platelets from the blood, platelet-rich plasma, or apheresis platelet;
(c) suspending the platelets in a buffer solution comprising trehalose;
(d) adding a stabilizing agent to the suspended platelets;
(e) cooling the platelets to a temperature of about −80° C.;
(f) storing the platelets at about −80° C. for a period of time;
(g) thawing the platelets at about 20-40° C.;
(h) isolating the thawed platelets;
(i) suspending the isolated thawed platelets in an activation buffer comprising BSA at about 1% by mass; and
(j) treating the recovered platelets with Thrombin Receptor Activating Peptide (TRAP; amino acid sequence: SFFLRN; SEQ ID NO: 1).

73. A kit for the detection of heparin-induced thrombocytopenia (HIT) antibodies in blood, plasma or serum, the kit comprising two or more of:
(a) cryopreserved platelets;
(b) one or more of platelet factor 4 (PF4; SEQ ID NO: 2) or heparin;
(c) one or more reagents for detecting platelet activation;
(d) optionally, buffers and receptacles; and
(e) optionally, one or more of packaging or instruction for use.

74. The kit of claim 73, wherein the one or more reagents for detecting platelet activation comprise reagents for the detection of thrombospondin-1.

75. The kit of claim 73, wherein the kit further comprises one or more negative control samples negative for platelet-activating pathogenic HIT antibodies; and one or more positive control samples positive for platelet-activating pathogenic HIT antibodies.

76. A method for treating or prophylaxis of hemorrhage in a subject in need thereof, the method comprising:
(a) obtaining cryopreserved platelets;
(b) thawing the cryopreserved platelets;
(c) contacting the subject with the thawed cryopreserved platelets.

77. The method of claim 76, wherein steps (a)-(b) comprise:
(i) obtaining blood, platelet-rich plasma, or apheresis-derived platelets from a second subject;
(ii) isolating platelets from the blood, platelet-rich plasma, or apheresis platelets;
(iii) suspending the platelets in a buffer solution comprising trehalose;
(iv) incubating trehalose-treated platelets at 37° C. for about 2 hours;
(v) adding a stabilizing agent to the suspended platelets;
(vi) cooling the platelets to a temperature of about −80° C.;
(vii) storing the platelets at about −80° C. for a period of time; and
(viii) thawing the platelets at about 20-40° C.

78. The method of claim 76, wherein steps (a)-(b) comprise:
(i) obtaining platelet-rich plasma or apheresis-derived platelets from a second subject;
(ii) adding trehalose to the platelet rich plasma or apheresis-derived platelets;
(iii) incubating trehalose-treated platelets at 37° C. for about 2 hours;
(iv) cooling the platelets to a temperature of about −80° C.;
(v) storing the platelets at about −80° C. for a period of time; and
(vi) thawing the platelets at about 20-40° C.

79. The method of claim 76, wherein the contacting in step (c) comprises applying the thawed cryopreserved platelets to an injury situs or parenterally administering the thawed cryopreserved platelets to the subject.

80. Use of cryopreserved platelets as a reagent, research tool, or as a medicament for treating a subject in need thereof.

81. Use of thawed cryopreserved platelets as a reagent, research tool, or as a medicament for treating a subject in need thereof.

82. Use of activated, thawed cryopreserved platelets as a reagent, research tool, or as a medicament for treating a subject in need thereof.
